(19) United States
(12) Patent Application Publication
(10) Pub. No.: US 2004/0071705 A1
(43) Pub. Date: Apr. 15, 2004

(54) Serum protein-associated target-specific ligands and identification method therefor

(75) Inventors: Aaron K. Sato, Somerville, MA (US); Albert Edge, Newton, MA (US)

Correspondence Address: FISH & RICHARDSON PC, 225 FRANKLIN ST, BOSTON, MA, 02110, US

(73) Assignee: DYAX CORPORATION

(21) Appl. No.: 10/602,141

(22) Filed: Jun. 23, 2003

Related U.S. Application Data
(60) Provisional application No. 60/390,657, filed on Jun. 21, 2002.

(51) Int. Cl. G01N 33/53; A61K 39/395

(52) U.S. Cl. 424/145.1; 435/7.1

(57) ABSTRACT

Disclosed is an artificial target-specific ligand that binds to both serum albumin and a particular molecular target. Interaction with serum albumin improves properties when administered to a subject. For example, an interaction between the ligand and serum albumin can extend the half-life of the ligand in circulation.

### CROSS-REFERENCE TO RELATED APPLICATIONS

This application claims priority to U.S. application Ser. No. 60/390,657, filed on Jun. 21, 2002, the contents of which are incorporated by reference in their entirety for all purposes.

### BACKGROUND

Serum albumin is an abundant transport protein of approximately 70 kilo-Daltons in circulating blood of mammalian species. For example, serum albumin is normally present at a concentration of approximately 3 to 4.5 grams per 100 ml of whole blood. Serum albumin provides several important functions in the circulatory system. For instance, it functions as a transporter of a variety of organic molecules found in the blood, as the main transporter of various metabolites such as fatty acids, hematin, and bilirubin, and, owing to its abundance, as an osmotic regulator of the circulating blood. It also has a broad affinity for small, negatively charged aromatic compounds. These binding functions enable serum albumin to serve as the principal carrier of fatty acids that are otherwise insoluble in circulating plasma.

Serum albumin can also bind to drugs that are administered to a subject. Indeed, one indicator of the efficacy of a drug is its affinity for serum albumin or other serum proteins. Binding to serum albumin can affect the overall distribution, metabolism, and bioavailability of many drugs.

It is known to conjugate drugs to serum albumin to extend their half-life and distribution. Chimeric albumin molecules such as HSA-CD4 and HSA-methotrexate have been utilized to increase the half-life and distribution of these potential therapeutics (see, e.g., Yeh et al. (1992) Proc. Natl. Acad. Sci. USA 89:1904-8 and Burger et al. (2001) Int. J Cancer 92:718).

### SUMMARY

In one aspect, the invention features a non-naturally occurring or isolated peptide (i) that interacts with (e.g., specifically binds to) a target (e.g., a target molecule, target cell, or target tissue) and that binds to a serum albumin (e.g., human serum albumin) and (ii), for example, has a half-life in vivo of greater than 30 minutes (or greater than 40, 60, 80, 120, 240 minutes, or greater than 5, 8, 12, 20, 24, or 36 hours) in a mouse model system. The affinity of the peptide for serum albumin can be less than its affinity for the target molecule. The K_{off} of the peptide for serum albumin can be faster than its K_{off} for the target molecule.

The half-life assessments in “mouse model system” are made by labeling the ligand with a radiolabel, injecting the labeled ligands into mice. The mice are sacrificed at different time points and serum collected from each time point. The amount of label in each sample is counted to generate a curve for ligand concentration vs. time. Half-life is determined by fitting the curve to the appropriate model. If the curve includes multiple phases, the half-life refers to the longest half-life that contributes to at least 15% of the amplitude of the curve. Of course, in an application of a method described herein, other methods and animals can be used to assess in vivo half-life.

The peptide can be made and/or identified by a method described herein.

The peptide can include one or more of the following exemplary features: an intra-molecular disulfide bond, a toxic moiety (e.g., cytotoxic moiety), a detectable label, a length of less than 32, 28, 24, 20, 18, or 16 residues, at least one aromatic amino acid (e.g., a di- or tri-peptide aromatic sequence). Cysteine residues in a peptide including a disulfide bond may be spaced by a loop of 4, 5, 6, 7, 8, 9, or 10, or more amino acids

The peptide may bind to the target molecule with a K_{D} less than 5, 2, 1, 0.5, 0.1, or 0.02 μM, or less than 1 0, 1, or 0.5 nM. The peptide may bind to the serum albumin with a K_{D} less than 50, 5, 2, 1, 0.5, 0.1, or 0.02 μM and/or greater than 0.1, 5, 20, or 50 nM, or 0.1, 0.5 or 1 μM. In an embodiment, the peptide binds with higher affinity to the target molecule than the serum molecule. For example, the K_{D} for binding the target molecule can be at least 2, 5, 10, 50, 100, 10³, or 10⁵ fold smaller (i.e., better) than the K_{D} for binding the serum albumin, or the fold preference can be, e.g., between 10 and 10⁷ fold, or 10-10³ fold.

In one embodiment, the peptide is not conjugated to another compound, e.g., another peptide or a non-biological polymer, e.g., a hydrophilic polymer it is not coupled to PEG. In another embodiment, the peptide is conjugated to a non-polymeric compound, e.g., a non-polymeric cytotoxin.

In one embodiment, the peptide and any conjugated compounds to which it is attached has a molecular weight of less than 4500, 4000, 3500, 3000, 2500, or 2000 Daltons.

In an embodiment, binding of the peptide to the target molecule and binding of the peptide to the serum albumin are mutually exclusive. In an embodiment, residues of the peptide that mediate binding to the target molecule and residues that mediate binding to the serum albumin are co-extensive. The peptide may include L- and/or D-amino acids. In another embodiment, binding of the peptide to the target molecule and binding of the peptide to the serum albumin can be concurrent.

In an embodiment, the target molecule includes an extracellular domain of a naturally occurring protein. The target molecule can include a mammalian, e.g., human protein, or fragment thereof. The target molecule is selected from the group consisting of CEA, VEGF-R2, an integrin subunit, and MUC1. In one embodiment, the peptide does not bind to VEGF-R2, e.g., the peptide is other than DX-954.

In one embodiment, the target molecule is not normally present in blood or serum. In one embodiment, the target molecule is not present on an endothelial cell. In another embodiment, the target molecule is present on an endothelial cell. In one embodiment, the target molecule is a cancer-specific antigen. In one embodiment, the target molecule is located in the lumen of a vesicle of other intracellular structure.

In one embodiment, the peptide is substantially free of a label, e.g., it is not covalently attached to a label. In one embodiment, the peptide is associated with a protein transduction domain (e.g., the HIV tat protein transduction domain) that enhances uptake of the peptide into a cell.

The peptide may be isolated by a method that includes screening a display library for members that display a molecule that binds to a serum albumin.

The invention also features an isolated nucleic acid that includes a sequence that encodes a polypeptide that includes the peptide that interacts with (e.g., specifically binds) to a target and that binds to a serum albumin. Also included are vectors and host cells containing the nucleic acid, e.g., vectors and host cells suitable for producing the nucleic acid molecule and/or the polypeptide.

In another aspect, the invention features a non-naturally occurring peptide (i) that specifically binds to a target molecule, other than a serum protein, and that binds to a serum protein (e.g., a serum protein other than serum albumin) with an affinity that is reduced relative to its affinity for the target molecule, and (ii) has a half-life in vivo of greater than 30 minutes (or greater than 40, 60, 80, 120, 240 minutes, or greater than 5, 8, 12, 20, 24, or 36 hours) in the mouse model system. The peptide may include other features described herein.

In still another aspect, the invention features a non-naturally occurring protein (i) that specifically binds to a target molecule, other than a serum protein, and that binds to a serum protein (e.g., a serum albumin) (e.g., with an affinity that is reduced relative to its affinity for the target molecule), and (ii) has a half-life in vivo of greater than 30 minutes (or greater than 40, 60, 80, 120, 240 minutes, or greater than 5, 8, 12, 20, 24, or 36 hours) in the mouse model system. The protein may include other features described herein. For example, the protein may include one or more immunoglobulin variable domains, e.g., two immunoglobulin variable domains (VL and VH). The immunoglobulin variable domain may bind to the target molecule and the serum protein by the CDRs. The protein may include other features described herein.

In one aspect, the invention features a method, e.g., a method of identifying a ligand that binds to a predetermined target and to a serum albumin. The method includes: providing a plurality of library members, each of which includes a diverse protein; and identifying one or a subset of members of the plurality which binds to both (1) a predetermined target, other than a serum albumin, and (2) a serum albumin, thereby identifying a ligand that binds to a predetermined target and to a serum albumin. The subset can include one, or at least one, two, five, ten, twenty, or fifty members. In one embodiment, the plurality of library members are each members of a display library, e.g., a cell or phage (e.g., filamentous phage) display library. In one embodiment, the library is arrayed, e.g., each member is disposed at a unique addressable location. The library can include at least 10³, 10⁵, 10⁶, 10⁷, or 10⁹ different members and optionally less than 10¹² or 10¹¹ different members.

In one embodiment, the identifying includes identifying of the first subset of the plurality, wherein each member of the first subset binds to the predetermined target, and identifying one or a subset of members of the first subset that bind to the serum albumin. In another embodiment, the identifying comprises identifying of the first subset of the plurality, wherein each member of the first subset binds to the serum albumin, and identifying one or a subset of members of the first subset that bind to the predetermined target. The identifying of the first subset can include contacting members of the library to the first compound and isolating members that interact with the first compound. The identifying a first subset and identifying a second subset each can include screening a display library. In another example, only some identifying steps include screening a display library. The first and/or second subset can include one, or at least one, two, five, ten, twenty, fifty, or a hundred members.

The target molecule can include a mammalian, e.g., human protein, or fragment thereof. The target molecule can be, for example, a target molecule mentioned herein, e.g., CEA, VEGF-R2, an integrin subunit, and MUC1. In one embodiment, the target molecule is a molecule other than a VEGF receptor, e.g., other than a VEGF-R2. In one embodiment, the particular target compound includes an extracellular domain of a naturally occurring protein. The target molecule can be used in a screen or selection in a cell free form or may be presented on a cell surface. In one embodiment, the target is a cell.

The method can further include assessing the in vivo half-life of one or more of the identified members. The method can further include formulating one or more of the identified members of the second subset as a pharmaceutical composition. The method can further include administering the pharmaceutical composition to a subject.

In one embodiment, each protein of the library includes an independent peptide binding domain, e.g., a peptide that includes a intramolecular disulfide bond or a linear peptide. In another embodiment, each protein of the library includes an immunoglobulin variable domain.

The method can further include mutagenizing an identified member, e.g., to create a second library of proteins. The method can be repeated with the second library of protein. In another example, the second library is screened with the first or second compound or for a physiological property, e.g., in vivo half-life.

One or more of the identified proteins can include a property described herein. For example, the protein may bind to the target molecule with a K_{D} less than 5, 2, 1, 0.5, 0.1, or 0.02 μM, or less than 10, 1, or 0.5 nM. The protein may bind to the serum albumin with a K_{D} less than 50, 5, 2, 1, 0.5, 0.1, or 0.02 μM and/or greater than 0.1, 5, 20, or 50 nM, or 0.1, 0.5 or 1 μM. In an embodiment, the identified protein binds with higher affinity to the target molecule than the serum molecule.

In an embodiment, binding of the protein to the target molecule and binding of the protein to the serum albumin are mutually exclusive. In an embodiment, residues of the protein that mediate binding to the target molecule and residues that mediate binding to the serum albumin are co-extensive.

The method can further include comparing the amino acid sequence of the members of the subset to each other to provide at least one profile.

In one embodiment, for each member of the plurality of library members, the diverse protein includes a diverse independent binding domain, e.g., a peptide binding domain that is less than 30, 28, 24, 20, 18, or 16 amino acids long. The peptide binding domain can include less than ten, six, five, or three constant positions, e.g., exactly two or no constant positions. The peptide binding domain can include one or more intramolecular disulfide bonds, e.g., a single disulfide bond. Between four and sixteen varied amino acids can be positioned between the constant cysteines that form a disulfide bond.

In another aspect, the invention features a method, e.g., a method of identifying a ligand that binds to a predetermined target and to a serum albumin. The method includes: (a) providing a plurality of library members, each of which includes a diverse protein; (b) identifying a subset of members of the plurality that binds to a predetermined target, other than serum albumin; (c) altering the sequence of at least one member of the subset to form an altered subset; and (d) identifying one or a subset of members of the altered subset which binds to a serum albumin, thereby identifying a ligand that binds to a predetermined target and to a serum albumin. A related method includes: (a) providing a plurality of library members, each of which includes a diverse protein; (b) identifying a subset of members of the plurality that binds to a serum albumin; (c) altering the sequence of at least one member of the subset to form an altered subset; and (d) identifying one or a subset of members of the altered subset which binds to a predetermined target, other than a serum albumin, thereby identifying ligand that binds to a predetermined target and to a serum albumin.

In one embodiment, the library is a display library, e.g., a cell or display library. In one embodiment, the library is arrayed. The identifying of the first subset can include contacting members of the library to the first compound and isolating members that interact with the first compound.

The identifying a first subset and identifying a second subset each can include screening a display library. In another example, only some identifying steps include screening a display library.

The target molecule can include a mammalian, e.g., human protein, or fragment thereof. The target molecule can be, for example, a target molecule mentioned herein, e.g., CEA, VEGF-R2, an integrin subunit, and MUC1. In one embodiment, the particular target compound includes an extracellular domain of a naturally occurring protein.

In one embodiment, the altered subset consists of variants of a plurality of members from the first identified subset, e.g., at least two, three, five, ten, twenty, fifty, or a hundred members. The altered subset can include at least 10³, 10⁵, 10⁶, 10⁷, or 10⁹ different members and optionally less than 10¹² or 10¹¹ different members.

The method can further include assessing the in vivo half-life of one or more second-identified members. The method can further include formulating one or more second-identified members as a pharmaceutical composition. The method can further include administering the pharmaceutical composition to a subject.

In one embodiment, each protein of the library includes an independent peptide binding domain, e.g., a peptide that includes a intramolecular disulfide bond or a linear peptide. In another embodiment, each protein of the library includes an immunoglobulin variable domain.

The method can further include mutagenizing a member identified from the second-identified subset, e.g., to create a second library of proteins. The method can be repeated with the second library of protein. In another example, the second library is screened with the first or second compound or for a physiological property, e.g., in vivo half-life.

One or more of the identified proteins can include a property described herein. For example, the protein may bind to the target molecule with a K_{D} less than 5, 2, 1, 0.5, 0.1, or 0.02 μM, or less than 10, 1, or 0.5 nM. The protein may bind to the serum albumin with a K_{D} less than 5, 2, 1, 0.5, 0.1, or 0.02 μM. In an embodiment, the identified protein binds with higher affinity to the target molecule than the serum molecule.

In an embodiment, binding of the protein to the target molecule and binding of the protein to the serum albumin are mutually exclusive. In an embodiment, residues of the protein that mediate binding to the target molecule and residues that mediate binding to the serum albumin are co-extensive.

In one embodiment, providing the altered subset comprises mutagenizing at least one member of the first-identified subset. In another embodiment, providing the altered subset comprises comparing amino acid sequences of members of the first-identified subset, inferring at least one profile for at least some of the members, and preparing the altered subset according to the at least one profile.

The method can include other features described herein.

In still another aspect, the invention features a method, e.g., a method of providing a candidate protein that binds to a target compound and to a serum albumin. The method includes: providing a library of diverse proteins; identifying, from the library, a member that binds to a target compound other than a serum albumin; determining, for the identified member, one or more amino acid positions that are non-essential for binding to the target compound or that are predicted as non-essential for binding to the target compound, modifying the one or more non-essential amino acid positions to provide a candidate protein; and evaluating binding of the candidate protein to a serum albumin. The method can further include evaluating binding of the candidate protein to the target compound. The method can further include evaluating at least a second candidate protein that is provided by the modifying.

In one embodiment, the evaluating includes contacting a plurality of candidate proteins provided by the modifying to immobilized serum albumin and identifying at least one candidate protein that interacts with the immobilized serum albumin.

The modifying can include making a substitution, deletions, or insertion. In one embodiment, the modifying includes varying the one or more non-essential amino acid positions using a set of amino acids, e.g., a set of at least three, five, ten, or twelve amino acids, or a set of amino acids that includes amino acids with aromatic side chains, e.g., tryptophan, tyrosine, and phenylalanine. For example, the modifying can include substituting at least one of the one or more non-essential amino acid positions with an aromatic side chain, e.g., tryptophan, tyrosine, or phenylalanine. In another embodiment the determining comprises alanine-scanning or aromatic amino acid scanning.

In one embodiment, the determining includes preparing a secondary library of variants, screening the secondary library to identify members that bind to the target molecule, and determining the amino acid sequence of members of the secondary library that bind to the target molecule.

In one embodiment, the determining further includes comparing the determined amino acid sequences to each other and/or to the amino acid sequence of the identified member.

The method can include other features described herein.

In one aspect, the invention features a method that includes: (a) providing a plurality of library members, each of which includes a diverse protein; (b) identifying a subset of members of the plurality that binds to a predetermined target, other than a given serum protein (e.g., serum albumin), or to the given serum protein; (c) altering the sequence of at least one member of the subset to form an altered subset; and (d) identifying one or a subset of members of the altered subset which binds to (1) the predetermined target if the identifying in (b) is to given serum protein or (2) the given serum protein, if the identifying in (b) is to the predetermined target, thereby identifying a target binding protein. The method can include other features described herein. The predetermined target can be a predetermined target compound, e.g., a proteinaceous compound, a predetermined cell, tissue, or organism or a predetermined particle, e.g., a virus or plaque. The predetermined cell can be, e.g., a cancer, or a cell of a pathogen.

In another aspect, the invention features a method of providing a target-binding protein that binds to a target (e.g., a target compound, or a target cell, tissue, or organ) and to serum albumin. The method includes: providing a library of diverse proteins; identifying, from the library, a plurality of members, wherein each member binds to a target other than a serum albumin; evaluating each member of the plurality for binding to serum albumin; and selecting a member of the plurality that binds to serum albumin, thereby providing a target-binding protein. For example, each member of the plurality is evaluated individually. In one embodiment, the target includes a cell, e.g., a mammalian cell or a pathogenic cell. The mammalian cell can be a diseased cell, e.g., a cancer cell.

In one embodiment, the library is a phage display library, and, for example, the evaluating comprises an ELISA assay that assessing binding of displaying phage to immobilized serum albumin. Results of the evaluating can be stored in a digital form. A subset of the results can be indicated to a user.

The method can include other features described herein.

In another aspect, the invention features a library of serum albumin-binding proteins. The library includes a plurality of proteins. Each protein of the plurality is substantially free of a functional immunoglobulin variable domain, and binds to a serum albumin with an affinity of at least 10 μM. For example, each protein of the plurality can include a peptide that independently binds to the serum albumin. In one embodiment, the peptide is less than 30, 28, 24, 20, 18, or 16 amino acids.

Proteins of the library may bind to serum albumin specifically or non-specifically. In an embodiment, at least one of the proteins of the plurality binds to serum albumin non-specifically.

In one embodiment, the library is a display library, e.g., a phage display or cell display library. In another embodiment, each protein of the library is immobilized at a discrete address on a surface.

In another aspect, the invention features a method of identifying a ligand that binds to a serum albumin and to a target molecule. The method includes: contacting a plurality of members of a library of serum-albumin binding proteins (e.g., a library described herein) to a selected target molecule; and identifying, from the plurality of members, one or more members that bind to the target molecule. The method can further include one or more of: formulating a functional segment of the one or more isolated members as a composition for administration to a subject; assessing the in vivo half-life of the one or more isolated members; determining the protein sequence of the isolated member or members of the isolated subset; producing a secondary library of variants of the one or more isolated members; screening the secondary library for one or more variant members that bind to the target molecule or a serum albumin. The method can include other features described herein.

In one aspect, the invention features a method, e.g., a method of identifying a ligand that binds to a predetermined target and to a serum protein. The method includes: providing a plurality of library members, each of which includes a diverse protein; and identifying one or a subset of members of the plurality which binds to both (1) a predetermined target, other than a serum protein, and (2) a serum protein, thereby identifying a ligand that binds to a predetermined target and to a serum protein. Examples of serum proteins include serum albumin, antibodies (e.g., IgG, IgM, and so forth), transferrin, a-macroglobulins, ferritin, apolipoproteins, transthyretin, protease inhibitors, retinol binding protein, thiostatin, a-fetoprotein, vitamin-D binding protein, and afamin. The method can include other features, e.g., as described above and elsewhere herein.

In still another aspect, the invention features a non-naturally occurring nucleic acid (e.g., a nucleic acid aptamer) that interacts with (e.g., specifically binds to) a target molecule, other than a serum protein, and that binds to a serum protein (e.g., a serum albumin) (e.g., with an affinity that is reduced relative to its affinity for the target molecule). The nucleic acid can have, e.g., an half-life in vivo of greater than 30 minutes (or greater than 40, 60, 80, 120, 240 minutes, or greater than 5, 8, 12, 20, 24, or 36 hours) in the mouse model system. The nucleic acid can have other features described herein. The invention can also be embodied using compounds that are not regular biological polymer. For example, compounds from any chemical library or collection can be screened using a method described herein to find a compound that interacts with a target molecule other than a serum protein and that also binds to a serum protein (e.g., serum albumin).

In still another aspect, the invention features a method of providing an agent. The method includes selecting an agent which has been tested for ability to bind to a target molecule and to a serum albumin, thereby providing the agent. For example, the agent is a peptide. The method can further include administering the agent to a subject. The selecting can include selecting for an extent of binding described herein, e.g., above or for a particular relative affinity, e.g., at least 1.5, 2, 5, 10, or 100 fold better binding to the target molecule. The method can include other features described herein.

In still another aspect, the invention features a method of treating a subject. The method includes providing (e.g., selecting) an agent which has been tested for ability to bind to a target molecule and to a serum albumin and administering the agent to the subject. For example, the agent is a peptide. The selecting can include selecting for an extent of binding described herein, e.g., above or for a particular relative affinity, e.g., at least 1.5, 2, 5, 10, or 100 fold better binding to the target molecule. The method can include other features described herein.

The term “polypeptide” refers to a polymer of three or more amino acids linked by a peptide bond. The polypeptide may include one or more unnatural amino acids. Typically, the polypeptide includes only natural amino acids. The term “peptide” refers to a polypeptide that is between three and thirty-two amino acids in length. A “protein” can include one or more polypeptide chains. A protein or polypeptide can also include one or more modifications, e.g., a glycosylation, amidation, prenylation, and so forth.

An “isolated composition” refers to a composition that is removed from at least 30% of at least one component of a natural sample from which the isolated composition can be obtained. Compositions may also be at least 50, 70, 75, 80, 90, 95, 98, or 99% isolated

“Binding affinity” refers to the apparent dissociation constant or K_{D}. A ligand may, for example, have a binding affinity of at least 10⁻⁵, 10⁻⁶, 10⁻⁷ or 10⁻⁸ M for a particular target molecule. Higher affinity binding of a ligand to a first target relative to a second target can be indicated by a smaller numerical value K_{D}¹ for binding the first target than the numerical value K_{D}² for binding the second target. In such cases the ligand has specificity for the first target relative to the second target. In exemplary cases, specific binding refers to binding of at least 2, 5, 10, 50, 100, or 1000 fold better for the desired target relative to a non-target. Variant specific binding refers to specific binding in cases where the non-target is at least 70, 80, or 90% identical to the desired target. A target-binding protein described herein can be a specific binding or a variant-specific binder. An interaction between a ligand described herein and serum albumin may or may not be specific, i.e., non-specific interactions can also be useful, e.g., for extending in vivo half-life. Typically, K_{D}'s are determined in PBS (phosphate buffered saline) at pH 7.2 unless otherwise indicated.

The term “diverse” refers to macromolecules that have one or more changes in sequence, e.g., nucleotide or amino acid changes, e.g., a substitution, insertion, or deletion.

The term “library” can be used to refer to any collection of at least two molecules, e.g., a library of nucleic acids or a library of polypeptides. Exemplary libraries can include at least 10², 10³, 10⁵, 10⁷ or 10⁹ unique members that are diverse with respect to each other.

The invention also includes sequences and variants thereof that include one or more substitutions, e.g., between one and six substitutions or at least one but less than 10, 5, 4, 3, 2, or 1% substituted. Whether or not a particular substitution will be tolerated, i.e., will not adversely affect desired biological properties, such as binding activity, can be determined by a functional test or by prediction, e.g., as described in Bowie, et al. (1990) Science 247:1306-1310. One or more or all substitutions may be conservative. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Still other substitutions, particularly in a synthetically produced peptide, may provide a non-naturally occurring amino acid.

All patent applications, patents, and references cited herein are incorporated by reference in their entirety.

### BRIEF DESCRIPTION OF THE DRAWING

FIG. 1 is a schematic of DX-1235. The solid lines indicate residues disposed in a cysteine loop. The upper amino acid sequence corresponds to DX-712 (SEQ ID NO: 2; see also Example 2, below). The lower amino acid sequence corresponds to DX-954 (SEQ ID NO: 1, see also Example 1, below). The line connecting the two cysteines in each amino acid sequence corresponds to a disulfide bond.

### DETAILED DESCRIPTION

In one aspect, an artificial target-specific ligand that binds to both serum albumin and a particular molecular target is created. Interaction with serum albumin may result in improved properties when administered to a subject. For example, an interaction between the ligand and serum albumin may extend the half-life of the ligand in circulation.

For example, binding of a small peptide ligand to serum albumin results in a larger effective molecular weight while circulating in the blood stream. The peptide uses its association with the larger serum albumin molecule to avoid clearance, e.g., in the kidney. However, the peptide remains effective in binding to its intended target as it can have a higher affinity for binding to the target molecule. In cases where the binding to serum albumin and to the target are mutually exclusive, localization of the serum albumin to the target is avoided.

### Identification of Ligands that Bind Serum Albumin and a Target

The following methods, among others, can be used to identify an artificial ligand that binds to both serum albumin and a particular molecular target.

1. In a first example, a library of peptides is screened for peptides that bind to a particular target. At an initial stage, the library of peptides can include diverse peptides that have a number of varied consecutive positions. Each position can be varied among a large set of amino acids (e.g., all twenty natural amino acids, natural amino acids in combination with one or more unnatural amino acids, or the nineteen non-cysteine amino acids). The initial identification of peptides that bind the target can include one or more rounds of screening against the target compound. The identified peptides are subsequently screened for binding to serum albumin, typically human serum albumin. Peptides that are identified in the subsequent screen are candidates for ligands that bind to both the particular target compound and serum albumin and are characterized further.

2. In a second example, an initial library of peptides is screened to identify peptides that bind to human serum albumin. Peptides so identified are then screened for binding against the target compound. Peptides identified in the second screen are candidates as ligands that bind to both the particular target compound and serum albumin and are characterized further.

3. In a third example, an initial library of peptides is screened to identify peptides that bind to a particular molecular target. The sequences of such peptides are characterized and a secondary library of peptides is constructed based on one or more peptides identified from the initial library. For example, the secondary library can be designed to retain an original residue with a frequency of at least 25, 50, or 75%. In other cases, the residue is allowed to vary, e.g., among all other possible amino acids. The secondary library is screened to identify peptides that bind to a serum albumin. Such peptides are further characterized.

4. In a fourth example, an initial library of peptides is screened to identify peptides that bind to a particular molecular target. The sequence of at least one such peptide is characterized and residues within the peptide that may be important for binding the target are identified. Such residues can be identified by a number of methods. For example, the identified peptides can be compared to each other to construct one or more consensus sequences. Positions that are conserved in the consensus are inferred to be essential for binding. In another example, the identified peptides are mutated, e.g., randomly or using a site-directed method such as alanine scanning. Functional variants of the peptides are sequenced to identify positions that are immutable or conserved. This latter case, variants that are non-functional provide direct evidence of the contribution of the varied residues.

A secondary library of peptides is constructed based on the above-information. In particular, the secondary library varies residues that are not essential for binding to the molecular target. Residues that are essential are either not varied (i.e., kept constant), or only varied among a limited set of amino acids (e.g., those that provide conserved substitutions). The secondary library is then screened to identify peptides that bind to a serum albumin.

5. In a fifth example, a library of peptides is screened for peptides that bind to a particular target. Peptides that are identified are then individually characterized, e.g., using a high-throughput platform described below. Each peptide is tested for binding to the particular target and to HSA. Information from the tests can be stored in a computer database which is then queried to identify peptides that are able to bind to both the target and to HSA.

6. In a sixth example, residues of a peptide that are non-essential for binding the particular molecular target are identified as described above. These residues are then systematically varied to include one or more aromatic amino acids or other motifs that are correlated with serum albumin binding. It is also possible to make a small library in which the non-essential residues are varied preferentially among aromatic amino acids. In other cases, a particular sequence such as Trp-Pro-Phe; Phe-Trp-Phe; Trp-Pro; Pro-Phe, or Tyr-Pro or a particular motif such as aromatic-proline-aromatic is included in the modified peptide.

7. In a seventh example, a peptide that binds to a particular molecular target is “tryptophan-scanned.” Variant peptides are made at each consecutive position such that the amino acid at that position is substituted with tryptophan. The binding affinity of the peptides for the particular molecular target and HSA are evaluated. In some cases, more than one peptide is found that is able to bind the target and HSA. In these cases, the tryptophan mutations might be combined to form a variant peptide with at least two substitutions.

In addition, any peptide identified as binding to a target and to HSA can be further mutagenized. Exemplary mutagenesis techniques include: error-prone PCR (Leung et al. (1989) Technique 1:11-15), recombination, DNA shuffling using random cleavage (Stemmer (1994) Nature 389-391), RACHITT™ (Coco et al. (2001) Nature Biotech. 19:354), site-directed mutagenesis (Zollner et al. (1987) Nucl Acids Res 10:6487-6504), cassette mutagenesis (Reidhaar-Olson (1991) Methods Enzymol. 208:564-586) and incorporation of degenerate oligonucleotides (Griffiths et al. (1994) EMBO J 13:3245).

Any of these methods are also readily extended to other proteins, e.g., variants of scaffold proteins described herein.

### A General Library of Serum Albumin Binders

As discussed above (e.g., in item 2 of “Library Screening”), it is possible to prepare a collection of peptides or proteins that bind to a serum albumin by screening an initial library for those members with this property. This collection can be replicated (e.g., by amplifying a display library or by synthesizing additional copies, e.g., of an array) to provide a general library of candidate serum for a number of different independent target molecules. The collection of peptides or proteins can also be provided as a kit, e.g., including instructions for use and/or reagents for screening.

A general library of serum albumin binders may also be produced, e.g., by determining a consensus sequence for serum albumin binding and synthesizing a collection of peptides or proteins that represent the diversity of the consensus. Such collections can be synthesized by generating nucleic acids encoding the respective peptide or proteins, e.g., as described below.

### Library Construction

A variety of methods are available to construct a library of peptides or other proteins (including polypeptides and oligomeric polypeptides). One exemplary method uses recombinant nucleic acid manipulation and expression, another, described below, uses protein arrays.

Recombinant Nucleic Acids. Nucleic acid libraries that encode a diverse set of peptides or other proteins are synthesized, typically, from synthetic oligonucleotides. These oligonucleotides can contain one or more degenerate positions such that, in the relevant frame for expression, different oligonucleotides of the population encode different amino acid sequences. In one implementation, the nucleic acid libraries are formed from degenerate oligonucleotide populations that include a distribution of nucleotides at each given position. The inclusion of a given sequence is random with respect to the distribution. One example of a degenerate source of synthetic diversity is an oligonucleotide that includes NNN wherein N is any of the four nucleotides in equal proportion.

Synthetic diversity can also be more constrained, e.g., to limit the number of codons in a nucleic acid sequence at a given trinucleotide to a distribution that is smaller than NNN. For example, such a distribution can be constructed using less than four nucleotides at some positions of the codon. A particular quadrant or sector of the genetic code can be selected by judicious choice of nucleotide subunits.

In addition, trinucleotide addition technology can be used to further constrain the distribution of diversity. So-called “trinucleotide addition technology” is described, e.g., in U.S. Pat. No. 5,869,644 and Vimekas et al. (1994) Nucl Acids Res 22:5600-7. Oligonucleotides are synthesized on a solid phase support, one codon (i.e., trinucleotide) at a time. The support includes many functional groups for synthesis such that many oligonucleotides are synthesized in parallel. The support is first exposed to a solution containing a mixture of the set of codons for the first position. The unit is protected so additional units are not added. The solution containing the first mixture is washed away and the solid support is deprotected so a second mixture containing a set of codons for a second position can be added to the attached first unit. The process is iterated to sequentially assemble multiple codons. Trinucleotide addition technology enables the synthesis of a nucleic acid that at a given position can encoded a selected number of amino acids. The frequency of these amino acids can be regulated by the proportion of codons in the mixture. Further, the choice of amino acids at the given position is not restricted to quadrants of the codon table as is the case if mixtures of single nucleotides are added during the synthesis. In some implementations, the set of selected codons corresponds to the extent of variation found in a profile of sequences (e.g., a profile of binders identified in a prior screen).

### Display Library Screening

Libraries of recombinant nucleic acids that encode a diverse set of proteins can be screened using a display library. A display library is a collection of entities; each entity includes an accessible polypeptide component and a recoverable component that encodes or identifies the peptide component. The polypeptide component can be of any length, e.g. from three amino acids to over 300 amino acids. In a selection, the polypeptide component of each member of the library is probed with the serum protein and if the polypeptide component binds to the protein, the display library member is identified, typically by retention on a support.

The screening of display libraries is advantageous, in that very large numbers (e.g., greater than 10⁵, 10⁷, or 5×10⁹) of potential binders can be tested, and successful binders isolated in a short period of time. Further, unlike immunization, ligands can be identified that bind to epitopes of serum proteins that are conserved among different species.

Retained display library members are recovered from the support and analyzed. The analysis can include amplification and a subsequent selection under similar or dissimilar conditions. For example, positive and negative selections can be alternated. The analysis can also include determining the amino acid sequence of the polypeptide component and purification of the polypeptide component for detailed characterization.

A variety of formats can be used for display libraries. Examples include the following.

Phage Display. One format utilizes viruses, particularly bacteriophages. This format is termed “phage display.” The peptide component is typically covalently linked to a bacteriophage coat protein. The linkage results form translation of a nucleic acid encoding the peptide component fused to the coat protein. The linkage can include a flexible peptide linker, a protease site, or an amino acid incorporated as a result of suppression of a stop codon. Phage display is described, for example, in Ladner et al., U.S. Pat. No. 5,223,409; Smith (1985) Science 228:1315-1317; WO 92/18619; WO 91/17271; WO 92/20791; WO 92/15679; WO 93/01288; WO 92/01047; WO 92/09690; WO 90/02809; de Haard et al. (1999) J. Biol. Chem 274:18218-30; Hoogenboom et al. (1998) Immunotechnology 4:1-20; Hoogenboom et al. (2000) Immunol Today 2:371-8; Fuchs et al. (1991) Bio/Technology 9:1370-1372; Hay et al. (1992) Hum Antibod Hybridomas 3:81-85; Huse et al. (1989) Science 246:1275-1281; Griffiths et al. (1993) EMBO J 12:725-734; Hawkins et al. (1992) J Mol Biol 226:889-896; Clackson et al. (1991) Nature 352:624-628; Gram et al. (1992) PNAS 89:3576-3580; Garrard et al. (1991) Bio/Technology 9:1373-1377; Rebar et al. (1996) Methods Enzymol. 267:129-49; Hoogenboom et al. (1991) Nuc Acid Res 19:4133-4137; and Barbas et al. (1991) PNAS 88:7978-7982.

Phage display systems have been developed for filamentous phage (phage f1, fd, and M13) as well as other bacteriophage (e.g. T7 bacteriophage and lambdoid phages; see, e.g., Santini (1998) J. Mol. Biol. 282:125-135; Rosenberg et al. (1996) Innovations 6:1-6; Houshmet al. (1999) Anal Biochem 268:363-370). The filamentous phage display systems typically use fusions to a minor coat protein, such as gene III protein, and gene VIII protein, a major coat protein, but fusions to other coat proteins such as gene VI protein, gene VII protein, gene IX protein, or domains thereof can also been used (see, e.g., WO 00/71694). In a preferred embodiment, the fusion is to a domain of the gene III protein, e.g., the anchor domain or “stump,” (see, e.g., U.S. Pat. No. 5,658,727 for a description of the gene III protein anchor domain). It is also possible to physically associate the protein being displayed to the coat using a non-peptide linkage, e.g., a non-covalent bond or a non-peptide covalent bond. For example, a disulfide bond and/or c-fos and c-jun coiled-coils can be used for physical associations (see, e.g., Crameri et al. (1993) Gene 137:69 and WO 01/05950).

The valency of the polypeptide component can also be controlled. Cloning of the sequence encoding the polypeptide component into the complete phage genome results in multivariant display since all replicates of the gene III protein are fused to the polypeptide component. For reduced valency, a phagemid system can be utilized. In this system, the nucleic acid encoding the polypeptide component fused to gene III is provided on a plasmid, typically of length less than 700 nucleotides. The plasmid includes a phage origin of replication so that the plasmid is incorporated into bacteriophage particles when bacterial cells bearing the plasmid are infected with helper phage, e.g. M13K01. The helper phage provides an intact copy of gene III and other phage genes required for phage replication and assembly. The helper phage has a defective origin such that the helper phage genome is not efficiently incorporated into phage particles relative to the plasmid that has a wild type origin.

Bacteriophage displaying the polypeptide component can be grown and harvested using standard phage preparatory methods, e.g. PEG precipitation from growth media.

After selection of individual display phages, the nucleic acid encoding the selected polypeptide components, by infecting cells using the selected phages. Individual colonies or plaques can be picked, the nucleic acid isolated and sequenced.

It is also possible to display multi-chain proteins such as Fab fragments on bacteriophage.

Cell-based Display. In still another format the library is a cell-display library. Proteins are displayed on the surface of a cell, e.g., a eukaryotic or prokaryotic cell. Exemplary prokaryotic cells include E. coli cells, B. subtilis cells, spores (see, e.g., Lu et al. (1995) Biotechnology 13:366). Exemplary eukaryotic cells include yeast (e.g., Saccharomyces cerevisiae, Schizosaccharomyces pombe, Hanseula, or Pichia pastoris). Yeast surface display is described, e.g., in Boder and Wittrup (1997) Nat. Biotechnol. 15:553-557 and WO 03/029456, which describes a yeast display system that can be used to display immunoglobulin proteins such as Fab fragments and the use of mating to generate combinations of heavy and light chains.

In one embodiment, variegate nucleic acid sequences are cloned into a vector for yeast display. The cloning joins the variegated sequence with a domain (or complete) yeast cell surface protein, e.g., Aga2, Aga1, Flo1, or Gas1. A domain of these proteins can anchor the polypeptide encoded by the variegated nucleic acid sequence by a transmembrane domain (e.g., Flo1) or by covalent linkage to the phospholipid bilayer (e.g., Gas1). The vector can be configured to express two polypeptide chains on the cell surface such that one of the chains is linked to the yeast cell surface protein. For example, the two chains can be immunoglobulin chains.

Ribosome Display. RNA and the polypeptide encoded by the RNA can be physically associated by stabilizing ribosomes that are translating the RNA and have the nascent polypeptide still attached. Typically, high divalent Mg²⁺ concentrations and low temperature are used. See, e.g., Mattheakis et al. (1994) Proc. Natl. Acad. Sci. USA 91:9022 and Hanes et al. (2000) Nat Biotechnol. 18:1287-92; Hanes et al. (2000) Methods Enzymol. 328:404-30. and Schaffitzel et al. (1999) J Immunol Methods. 231(1-2):119-35.

Peptide-Nucleic Acid Fusions. Another format utilizes peptide-nucleic acid fusions. Polypeptide-nucleic acid fusions can be generated by the in vitro translation of mRNA that include a covalently attached puromycin group, e.g., as described in Roberts and Szostak (1997) Proc. Natl. Acad. Sci. USA 94:12297-12302, and U.S. Pat. No. 6,207,446. The mRNA can then be reverse transcribed into DNA and crosslinked to the polypeptide.

Other Display Formats. Yet another display format is a non-biological display in which the polypeptide component is attached to a non-nucleic acid tag that identifies the polypeptide. For example, the tag can be a chemical tag attached to a bead that displays the polypeptide or a radiofrequency tag (see, e.g., U.S. Pat. No. 5,874,214).

### Synthetic Peptides

The binding ligand can include an artificial peptide of 32 amino acids or less, that independently binds to a target molecule. Some synthetic peptides can include one or more disulfide bonds. Other synthetic peptides, so-called “linear peptides,” are devoid of cysteines. Synthetic peptides may have little or no structure in solution (e.g., unstructured), heterogeneous structures (e.g., alternative conformations or “loosely structured), or a singular native structure (e.g., cooperatively folded). Some synthetic peptides adopt a particular structure when bound to a target molecule. Some exemplary synthetic peptides are so-called “cyclic peptides” that have at least disulfide bond, and, for example, a loop of about 4 to 12 non-cysteine residues. Many exemplary peptides are less than 28, 24, 20, or 18 amino acids in length.

Peptide sequences that independently bind a molecular target can be selected from a display library or an array of peptides. After identification, such peptides can be produced synthetically or by recombinant means. The sequences can be incorporated (e.g., inserted, appended, or attached) into longer sequences.

The following are some exemplary phage libraries that can be screened to find at least some of the polypeptide ligands described herein. Each library displays a short, variegated exogenous peptide on the surface of M13 phage. The peptide display of five of the libraries was based on a parental domain having a segment of 4, 5, 6, 7, 8, 10, 11, or 12 amino acids, respectively, flanked by cysteine residues. The pairs of cysteines are believed to form stable disulfide bonds, yielding a cyclic display peptide. The cyclic peptides are displayed at the amino terminus of protein III on the surface of the phage. The libraries were designated TN6/7, TN7/4, TN8/9, TN9/4, TN10/10. TN11/1, and TN12/1. A phage library with a 20-amino acid linear display was also screened; this library was designated Lin20.

The TN6/7 library was constructed to display a single cyclic peptide contained in a 12-amino acid variegated template. The TN6/6 library utilized a template sequence of Xaa₁-Xaa₂-Xaa₃-Cys₄-Xaa₅-Xaa₆-Xaa₇-Xaa₈-Cys₉-Xaa₁₀-Xaa₁₁-Xaa₁₂ (SEQ ID NO: 5), where each variable amino acid position in the amino acid sequence of the template is indicated by a subscript integer. Each variable amino acid position (Xaa) in the template was varied to contain any of the common a-amino acids, except cysteine (Cys).

The TN7/4 library was constructed to display a single cyclic peptide contained in a 12-amino acid variegated template. The TN7/4 library utilized a template sequence of Xaa₁-Xaa₂-Xaa₃-Cys₄-Xaa₅-Xaa₆-Xaa₇-Xaa₈-Xaa₉-Cys₁₀-Xaa₁₁-Xaa₁₂-Xaa₁₃ (SEQ ID NO: 6), where each variable amino acid position in the amino acid sequence of the template is indicated by a subscript integer. Each variable amino acid position (Xaa) in the template was varied to contain any of the common α-amino acids, except cysteine (Cys).

The TN8/9 library was constructed to display a single binding loop contained in a 14-amino acid template. The TN8/9 library utilized a template sequence of Xaa₁-Xaa₂-Xaa₃-Cys-Xaa₅-Xaa₆-Xaa₇-Xaa₈-Xaa₉-Xaa₁₀-Cys-Xaa₁₂-Xaa₁₃-Xaa₁₄ (SEQ ID NO: 7). Each variable amino acid position (Xaa) in the template were varied to permit any amino acid except cysteine (Cys).

The TN9/4 library was constructed to display a single binding loop contained in a 15-amino acid template. The TN9/4 library utilized a template sequence Xaa₁-Xaa₂-Xaa₃-Cys₄-Xaa₅-Xaa₆-Xaa₇-Xaa₈-Xaa₉-X₁₀-Xaa₁₁-Cys₁₂-Xaa₁₃-Xaa₁₄-Xaa₁₅ (SEQ ID NO: 8). Each variable amino acid position (Xaa) in the template were varied to permit any amino acid except cysteine (Cys).

The TN10/10 library was constructed to display a single cyclic peptide contained in a 16-amino acid variegated template. The TN10/9 library utilized a template sequence Xaa₁-Xaa₂-Xaa₃-Cys₄-Xaa₅-Xaa₆-Xaa₇-Xaa₈-Xaa₉-Xaa₁₀-Xaa₁₁-Xaa₁₂-Cys₁₃-Xaa₁₄-Xaa₁₅-Xaa₁₆ (SEQ ID NO: 9), where each variable amino acid position in the amino acid sequence of the template is indicated by a subscript integer. Each variable amino acid position (Xaa) was to permit any amino acid except cysteine (Cys).

The TN11/1 library was constructed to display a single cyclic peptide contained in a 17-amino acid variegated template. The TN11/1 library utilized a template sequence Xaa₁-Xaa₂-Xaa₃-Cys₄-Xaa₅-Xaa₆-Xaa₇-Xaa₈-Xaa₉-Xaa₁₀-Xaa₁₁-Xaa₁₂-Xaa₁₃-Cys₁₄-Xaa₁₅-Xaa₁₆-X₁₇ (SEQ ID NO: 10), where each variable amino acid position in the amino acid sequence of the template is indicated by a subscript integer. Each variable amino acid position (Xaa) was to permit any amino acid except cysteine (Cys).

The TN12/1 library was constructed to display a single cyclic peptide contained in an 18-amino acid template. The TN12/1 library utilized a template sequence Xaa₁-Xaa₂-Xaa₃-Cys₄-Xaa₅-Xaa₆-Xaa₇-Xaa₈-Xaa₉-Xaa₁₀-Xaa₁₁-Xaa₁₂-Xaa₁₃-Xaa₁₄-Cys₁₅-Xaa₁₆-Xaa₁₇-Xaa₁₈ (SEQ ID NO: 11), where each variable amino acid position in the amino acid sequence of the template is indicated by a subscript integer. The amino acid positions Xaa₁, Xaa₂, Xaa₁₇ and Xaa₁₈ of the template were varied, independently, to permit each amino acid selected from the group of 12 amino acids consisting of Ala, Asp, Phe, Gly, His, Leu, Asn, Pro, Arg, Ser, Trp, and Tyr. The amino acid positions Xaa₃, Xaa₅, Xaa₆, Xaa₇, Xaa₈, Xaa₉, Xaa₁₀, Xaa₁₁, Xaa₁₂, Xaa₁₃, Xaa₁₄, Xaa₁₆ of the template were varied, independently, to permit any amino acid except cysteine (Cys).

The Lin20 library was constructed to display a single linear peptide in a 20-amino acid template. The amino acids at each position in the template were varied to permit any amino acid except cysteine (Cys).

The techniques discussed in Kay et al., Phage Display of Peptides and Proteins: A Laboratory Manual (Academic Press, Inc., San Diego 1996) and U.S. Pat. No. 5,223,409 are useful for preparing a library of potential binders corresponding to the selected parental template. The libraries described above can be prepared according to such techniques, and screened, e.g., as described above, for peptides that bind to a serum albumin and a particular molecular target.

For any particular peptide that includes an intra-molecular disulfide bond, the peptide can be redesigned to replace the disulfide bond that maintains the geometry of the loop. For example, the distance between the alpha carbons of the first amino acid of the loop (which is C-terminal to the first cysteine of the loop) and the last amino acid of the loop (which is N-terminal to the second cysteine of the loop) can be maintain within 10, 6, 4, or 3 Angstroms of the distance between those alpha carbons in a disulfide bonded loop. In another example, the alpha carbons of the first amino acid of the loop and the last amino acid of the loop are maintained within 15, 12, 10, 8, or 7 inter-atomic bonds of each other. It is also possible to position another amino acid (natural or non-natural) in place of the cysteines, in which case the alpha carbons of these respective replacement amino acids may be within 9, 8, or 6 bonds of each other. Exemplary bonds include C—C, C—N, C—S, O—N, and C—O bonds. Generally, any chemical linker of appropriate length can be used to replace a disulfide bond.

### Other Exemplary Scaffolds

Other exemplary scaffolds that can be variegated to produce a protein that binds to serum albumin and a particular target can include: extracellular domains (e.g., fibronectin Type III repeats, EGF repeats); protease inhibitors (e.g., Kunitz domains, ecotin, BPTI, and so forth); TPR repeats; trifoil structures; zinc finger domains; DNA-binding proteins; particularly monomeric DNA binding proteins; RNA binding proteins; enzymes, e.g., proteases (particularly inactivated proteases), RNase; chaperones, e.g., thioredoxin, and heat shock proteins; and intracellular signaling domains (such as SH2 and SH3 domains) and antibodies (e.g., Fab fragments, single chain Fv molecules (scFV), single domain antibodies, camelid antibodies, and camelized antibodies); T-cell receptors and MHC proteins.

U.S. Pat. No. 5,223,409 also describes a number of so-called “mini-proteins,” e.g., mini-proteins modeled after α-conotoxins (including variants GI, GII, and MI), mu-(GIIIA, GIIIB, GIIIC) or OMEGA-(GVIA, GVIB, GVIC, GVIIA, GVIIB, MVIIA, MVIIB, etc.) conotoxins.

In many embodiments, the scaffold may be less than 50 amino acids in length. In some cases, a ligand, based on the scaffold, binds to a target molecule on one particular surface, whereas a different, non-overlapping surface binds to serum albumin. In other cases, the binding interface for the target and the serum albumin are co-extensive or at least partially overlapping. For example, binding by the ligand to the target may exclude binding to serum albumin. This configuration, for example, prevents localization of serum albumin to the vicinity of the target molecule.

### Antibody Display Libraries

It may also be possible to identify immunoglobulin proteins (including antibodies, Fab's, scFv's, camelids, and other antibody derivatives) that bind to a particular target compound and to serum albumin. For example, immunoglobulin proteins that have CDRs that bind to both a particular target compound and to serum albumin can be identified, e.g., using a display library.

In one implementation, an antibody library is screened as described above for peptide libraries. Such screens can include two or more sequential screens, e.g., first for antibodies that bind to a target protein, and then for antibodies so-identified that also bind to serum albumin. In another implementation, the amino acid sequences of the target protein and HSA are compared to identify peptides that are similar, e.g., include, at at least 50% of the residues, conserved substitutions or at least 20, 40, 50, or 60% identity. The peptide may be, e.g., between 6 and 32, 6 and 20, or 8 and 15 amino acids in length.

Antibodies are then identified that bind to such peptides, e.g., to the peptide derived from the target protein that has sequence similarity to HSA. For example, an antibody library may be screened using such a peptide as a target or the larger target protein as a target (in which case the peptide may be used to elute relevant antibodies). In another example, an animal is immunized with such a peptide, and antibodies from the animal are isolated.

Antibody derivatives, e.g., derivatives substantially free of an Fc region, may be similarly isolated or may be prepared, e.g., by modification of a full-length antibody. Such derivatives may have extended half-lives in vivo as a result of their association with serum albumin.

A typical antibody display library displays a polypeptide that includes a VH domain and a VL domain. An “immunoglobulin domain” refers to a domain from the variable or constant domain of immunoglobulin molecules. Immunoglobulin domains typically contain two β-sheets formed of about seven β-strands, and a conserved disulphide bond (see, e.g., A. F. Williams and A. N. Barclay 1988 Ann. Rev Immunol. 6:381-405). The display library can display the antibody as a Fab fragment (e.g., using two polypeptide chains) or a single chain Fv (e.g., using a single polypeptide chain). Other formats can also be used. The domains can be completely, or at least partially human.

As in the case of the Fab and other formats, the displayed antibody can include a constant region as part of a light or heavy chain. In one embodiment, each chain includes one constant region, e.g., as in the case of a Fab. In other embodiments, additional constant regions are displayed.

Antibody libraries can be constructed by a number of processes (see, e.g., de Haard et al. (1999) J. Biol. Chem 274:18218-30; Hoogenboom et al. (1998) Immunotechnology 4:1-20. and Hoogenboom et al. (2000) Immunol Today 21:371-8. Further, elements of each process can be combined with those of other processes. The processes can be used such that variation is introduced into a single immunoglobulin domain (e.g., VH or VL) or into multiple immunoglobulin domains (e.g., VH and VL). The variation can be introduced into an immunoglobulin variable domain, e.g., in the region of one or more of CDR1, CDR2, CDR3, FR1, FR2, FR3, and FR4, referring to such regions of either and both of heavy and light chain variable domains. In one embodiment, variation is introduced into all three CDRs of a given variable domain. In another preferred embodiment, the variation is introduced into CDR1 and CDR2, e.g., of a heavy chain variable domain. Any combination is feasible. In one process, antibody libraries are constructed by inserting diverse oligonucleotides that encode CDRs into the corresponding regions of the nucleic acid. The oligonucleotides can be synthesized using monomeric nucleotides or trinucleotides. For example, Knappik et al. (2000) J. Mol. Biol. 296:57-86 describe a method for constructing CDR encoding oligonucleotides using trinucleotide synthesis and a template with engineered restriction sites for accepting the oligonucleotides.

In yet another process, antibody libraries are constructed from nucleic acid amplified from naive germline immunoglobulin genes or from somatically mutated immunoglobulin genes. The amplified nucleic acid includes nucleic acid encoding the VH and/or VL domain. Sources of immunoglobulin-encoding nucleic acids are described below. Amplification can include PCR, e.g., with primers that anneal to the conserved constant region, or another amplification method.

### Screening Phage Display Libraries for Serum Protein Binding Peptides

In a typical screen, a phage library is contacted with and allowed to bind the target compound or a fragment thereof. To facilitate separation of binders and non-binders in the screening process, it is often convenient to immobilize the target compound on a solid support, although it is also possible to first permit binding to the target compound in solution and then segregate binders from non-binders by coupling the target compound to a support. By way of illustration, when incubated in the presence of the target, phage bearing a target-binding moiety form a complex with the target compound immobilized on a solid support whereas non-binding phage remain in solution and may be washed away with buffer. Bound phage may then be liberated from the target by a number of means, such as changing the buffer to a relatively high acidic or basic pH (e.g., pH 2 or pH 10), changing the ionic strength of the buffer, adding denaturants, or other known means.

For example to identify HSA-binding ligands, purified HSA or whole serum can be adsorbed (by passive immobilization) to a solid surface, such as the plastic surface of wells in a multi-well assay plate. In the case of using whole serum, the HSA that is bound may be associated with natural compounds, e.g., fatty acids. Subsequently, an aliquot of a phage display library was added to a well under appropriate conditions that maintain the structure of the immobilized HSA and the phage, such as pH 6-7. Phage in the libraries that display peptide loop structures that bind the immobilized HSA are retained bound to the HSA adhering to the surface of the well and non-binding phage can be removed. Since both specific and non-specific binding interactions may be useful, it may or may not be necessary to include a blocking agent during the binding of the phage library to the immobilized HSA.

Phage bound to the immobilized HSA may then be eluted by washing with a buffer solution having a relatively strong acid pH (e.g., pH 2) or an alkaline pH (e.g., pH 8-9). The solutions of recovered phage that are eluted from the HSA are then neutralized and may, if desired, be pooled as an enriched mixed library population of phage displaying serum albumin binding peptides. Alternatively the eluted phage from each library may be kept separate as a library-specific enriched population of HSA binders. Enriched populations of phage displaying serum albumin binding peptides may then be grown up by standard methods for further rounds of screening and/or for analysis of peptide displayed on the phage and/or for sequencing the DNA encoding the displayed binding peptide.

One of many possible alternative screening protocols uses HSA target molecules that are biotinylated and that can be captured by binding to streptavidin, for example, coated on particles. As is described in an example below, phage displaying HSA binding peptides were selected from a library in such a protocol in which phage displaying HSA binding peptides were bound to a caprylate-biotinylated-HSA in solution at pH 7.4 in phosphate buffered saline (PBS) supplemented with 0.1% Tween 20 nonionic detergent and also 0.1% sodium caprylate, which is known to stabilize HSA against temperature-induced denaturation and proteolytic attack. The caprylate-biotinylated-HSA/phage complexes in solution were then captured on streptavidin-coated magnetic beads. Phage were subsequently eluted from the beads for further study.

Recovered phage may then be amplified by infection of bacterial cells, and the screening process may be repeated with the new pool of phage that is now depleted in non-HSA binders and enriched in HSA binders. The recovery of even a few binding phage may be sufficient to carry the process to completion. After a few rounds of selection, the gene sequences encoding the binding moieties derived from selected phage clones in the binding pool are determined by conventional methods, revealing the peptide sequence that imparts binding affinity of the phage to the target. An increase in the number of phage recovered after each round of selection and the recovery of closely related sequences indicate that the screening is converging on sequences of the library having a desired characteristic.

After a set of binding polypeptides is identified, the sequence information may be used to design other, secondary libraries, biased for members having additional desired properties.

Other types of display libraries can be used to identify an HSA binder.

Display technology can also be used to obtain ligands that are specific to particular epitopes of a target. This can be done, for example, by using competing non-target molecules that lack the particular epitope or are mutated within the epitope, e.g., with alanine. Such non-target molecules can be used in a negative selection procedure as described below, as competing molecules when binding a display library to the target, or as a pre-elution agent, e.g., to capture in a wash solution dissociating display library members that are not specific to the target.

The binding properties of a ligand that binds a serum albumin can be readily assessed using various assay formats. For example, the binding property of a ligand can be measured in solution by fluorescence anisotropy, which provides a convenient and accurate method of determining a dissociation constant (K_{D}) of a binding moiety for a serum albumin from one or more different species. In one such procedure, a binding moiety described herein is labeled with fluorescein. The fluorescein-labeled binding moiety may then be mixed in wells of a multi-well assay plate with various concentrations of a particular species of serum albumin. Fluorescence anisotropy measurements are then carried out using a fluorescence polarization plate reader. The binding interaction between a serum albumin and a ligand can be similarly characterized. Other solution measures for studying binding properties include fluorescence resonance energy transfer (FRET) and NMR.

Binding properties can also be characterized using a method wherein one binding partner is immobilized. Such methods include ELISA and surface plasmon resonance.

### Protein Arrays

Arrays of peptides can be produced. Members of a library of peptides are disposed at discrete positions on an array (e.g., a planar array). A single species of peptide or a pool can be located at each position. The array is contacted with a target molecule or a serum albumin and positions on the array that are bound by the target and/or by the serum albumin are identified, e.g., by direct or indirect labeling.

In addition, peptides can be directly synthesized on the array. For example, U.S. Pat. No. 5,143,854 provides a photolithographic method of producing an array of peptides or proteins. This method does not require synthesizing nucleic acids encoding the peptides or proteins. The peptides can be made from L- or D-amino acids.

Additional methods of producing protein arrays are described, e.g., in De Wildt et al. (2000) Nat. Biotechnol. 18:989-994; Lueking et al. (1999) Anal. Biochem. 270:103-111; Ge (2000) Nucleic Acids Res. 28, e3, I-VII; MacBeath and Schreiber (2000) Science 289:1760-1763; WO 0/98534, WO01/83827, WO02/12893, WO 00/63701, WO 01/40803 and WO 99/51773. In some implementations, polypeptides (including peptides) are spotted onto discrete addresses of the array, e.g., at high speed, e.g., using commercially available robotic apparati, e.g., from Genetic MicroSystems or BioRobotics. The array substrate can be, for example, nitrocellulose, plastic, glass, e.g., surface-modified glass. The array can also include a porous matrix, e.g., acrylamide, agarose, or another polymer.

### Serum Binding Protein Ligand Variants

It is also possible to use a variant of a serum binding protein ligand described herein or isolated by a method described herein. A number of variants are possible. A variant can be prepared and then tested, e.g., using a binding assay described above (such as fluorescence anisotropy). If the variant is function, it can be used as an affinity reagent to isolate a serum protein and associated compounds.

One type of variant is a truncation of a ligand described herein or isolated by a method described herein. In this example, the variant is prepared by removing one or more amino acid residues of the ligand can be removed from the N or C terminus. In some cases, a series of such variants is prepared and tested. Information from testing the series is used to determine a region of the ligand that is essential for binding the serum protein. A series of internal deletions or insertions can be similarly constructed and tested.

Another type of variant is a substitution. In one example, the ligand is subjected to alanine scanning to identify residues that contribute to binding activity. In another example, a library of substitutions at one or more positions is constructed. The library may be unbiased or, particularly if multiple positions are varied, biased towards an original residue. In some cases, the substations are limited to conservative substitutions.

A related type of variant is a ligand that includes one or more non-naturally occurring amino acids. Such variant ligands can be produced by chemical synthesis. One or more positions can be substituted with a non-naturally occurring amino acid. In some cases, the substituted amino acid may be chemically related to the original naturally occurring residue (e.g., aliphatic, charged, basic, acidic, aromatic, hydrophilic) or an isostere of the original residue.

It may also be possible to include non-peptide linkages and other chemical modification. For example, part or all of the ligand may be synthesized as a peptidomimetic, e.g., a peptoid (see, e.g., Simon et al. (1992) Proc. Natl. Acad. Sci. USA 89:9367-71 and Horwell (1995) Trends Biotechnol.13:132-4). A peptide may include one or more (e.g., all) non-hydrolyzable bonds. Many non-hydrolyzable peptide bonds are known in the art, along with procedures for synthesis of peptides containing such bonds. Exemplary non-hydrolyzable bonds include —[CH₂NH]— reduced amide peptide bonds, —[COCH₂]— ketomethylene peptide bonds, —[CH(CN)NH]—(cyanomethylene)amino peptide bonds, —[CH₂CH(OH)]— hydroxyethylene peptide bonds, —[CH₂O]—peptide bonds, and —[CH₂S]— thiomethylene peptide bonds (see e.g., U.S. Pat. No. 6,172,043).

### Automated Methods and Information Management

Any and all aspects of the ligand screening platform can be automated. Automation, for example, can be used to process multiple different samples automatically. Liquid handling units can be used to isolate compounds that bind to serum albumin and to a target molecule and can automatically subject the isolated compounds to analytical methods. Automation can also be used to produce and test ligands.

Equipment. Various robotic devices can be employed in the automation process. These include multi-well plate conveyance systems, magnetic bead particle processors, and liquid handling units. These devices can be built on custom specifications or purchased from commercial sources, such as Autogen (Framingham Mass.), Beckman Coulter (USA), Biorobotics (Woburn Mass.), Genetix (New Milton, Hampshire UK), Hamilton (Reno Nev.), Hudson (Springfield N.J.), Labsystems (Helsinki, Finland), Packard Bioscience (Meriden Conn.), and Tecan (Mannedorf, Switzerland).

Information Management. Information generated by the ligand-screening platform can be stored in a computer database (e.g., in digital form). This information can include information that describes the binding properties of a potential ligand for one or more compounds, e.g. for the target compound, for a serum albumin, and for a non-target compound. Examples of non-target compounds include compounds that are homologous, yet non-identical to the target. Such compounds may be present on different cells, e.g., non-target cells. For example, the database can include information that describes a property of an associated compound (e.g., protein sequence, chemical structure, abundance, modification state, etc. and information that describes the sample (e.g., identity of its source, date, processing method, pathology, treatment, etc.). These items of information can be associated with each other. For example, a query about a particular state, e.g., a particular disease or treatment, can be used to identify properties of associated compounds found in that state. Likewise, a particular property of one or more associated compounds can be used as a query to identify states with which the property is prevalent.

The database can also be used to analyze one or more sequenced HSA-binders or target-binders. The sequences can be compared to each other, e.g., to generate a consensus or profile that may indicate positions that are important for binding. Software can be used to compare profiles or to produce structural models from the profiles.

The database server can also be configured to communicate with each device using commands and other signals that are interpretable by the device. The computer-based aspects of the system can be implemented in digital electronic circuitry, or in computer hardware, firmware, software, or in combinations thereof. An apparatus of the invention, e.g., the database server, can be implemented in a computer program product tangibly embodied in a machine-readable storage device for execution by a programmable processor; and method actions can be performed by a programmable processor executing a program of instructions to perform functions described herein by operating on input data and generating output. One non-limiting example of an execution environment includes computers running Windows NT 4.0 (Microsoft) or better or Solaris 2.6 or better (Sun Microsystems) operating systems.

The invention also features machine-readable software or instructions which enable an apparatus to produce a ligand (e.g., a peptide) described herein.

### High-Throughput Ligand Discovery

One exemplary high-throughput ligand discovery method includes screening a phage display library that has a diversity library of at least 10⁷ or 10⁸. Phage are contacted to a target molecule, e.g., immobilized on a magnetic bead. Binding phage are isolated, amplified and rescreened in one or more additional cycles. Then individual phage are isolated, e.g., into wells of a microtitre plate, and characterized.

For example, robots can be used to set up two ELISA assays for each individual phage. One assay is for binding to the particular target molecule, the other is for binding to a serum albumin. An automated plate reader can evaluate the assays and communicate results to a computer system that stores the results in an accessible format, e.g., in a database, spread sheet, or word processing document. Results are analyzed to identify phage that display a protein that binds to both the particular target and to the serum albumin. Results can be further sorted, e.g., by affinity or relative affinity, e.g., to identify proteins that bind with higher affinity to the target than to the albumin.

### Exemplary Targets

Generally, any molecular species can be used as a target. In some embodiment, more than one species is used as a target, e.g., a sample is exposed to a plurality of targets. The target can be of a small molecule (e.g., a small organic or inorganic molecule), a polypeptide, a nucleic acid, cells, and so forth.

One class of targets includes polypeptides. Examples of such targets include small peptides (e.g., about 3 to 30 amino acids in length), single polypeptide chains, and multimeric polypeptides (e.g., protein complexes).

A polypeptide target can be modified, e.g., glycosylated, phosphorylated, ubiquitinated, methylated, cleaved, disulfide bonded and so forth. Preferably, the polypeptide has a specific conformation, e.g., a native state or a non-native state. In one embodiment, the polypeptide has more than one specific conformation. For example, prions can adopt more than one conformation. Either the native or the diseased conformation can be a desirable target, e.g., to isolate agents that stabilize the native conformation or that identify or target the diseased conformation. In one embodiment, the ligand binds to the target only in a particular conformation. Certain conformations can be stabilized, e.g., using a disulfide bond.

In some cases, however, the polypeptide is unstructured, e.g., adopts a random coil conformation or lacks a single stable conformation. Agents that bind to an unstructured polypeptide can be used to identify the polypeptide when it is denatured, e.g., in a denaturing SDS-PAGE gel, or to separate unstructured isoforms of the polypeptide for correctly folded isoforms, e.g., in a preparative purification process.

Some exemplary polypeptide targets include: cell surface proteins (e.g., glycosylated surface proteins or hypoglycosylated variants), cancer-associated proteins, cytokines, chemokines, peptide hormones, neurotransmitters, cell surface receptors (e.g., cell surface receptor kinases, seven transmembrane receptors, virus receptors and co-receptors, extracellular matrix binding proteins such as integrins, cell-binding proteins (e.g., cell attachment molecules or “CAMs” such as cadherins, selectins, N-CAM, E-CAM, U-CAM, I-CAM and so forth), or a cell surface protein (e.g., of a mammalian cancer cell or a pathogen). In some embodiments, the polypeptide is associated with a disease, e.g., cancer.

The target polypeptide is preferably soluble. For example, soluble domains or fragments of a protein can be used. This option is particularly useful for identifying molecules that bind to transmembrane proteins such as cell surface receptors and retroviral surface proteins. In one embodiment, the target molecule is a protein that is not normally present in a particular environment unless the subject has a disease or disorder.

Some exemplary targets include: cell surface proteins (e.g., glycosylated surface proteins or hypoglycosylated variants), cancer-associated proteins, cytokines, chemokines, peptide hormones, neurotransmitters, cell surface receptors (e.g., cell surface receptor kinases, seven transmembrane receptors, virus receptors and co-receptors, extracellular matrix binding proteins, cell-binding proteins, antigens of pathogens (e.g., bacterial antigens, malarial antigens, and so forth).

More specific examples include: integrins, cell attachment molecules or “CAMs” such as cadherins, selections, N-CAM, E-CAM, U-CAM, I-CAM and so forth); proteases, e.g., subtilisin, trypsin, chymotrypsin; a plasminogen activator, such as urokinase or human tissue-type plasminogen activator (t-PA); bombesin; factor IX, thrombin; CD-4; CD-19; CD20; platelet-derived growth factor; insulin-like growth factor-I and -II; nerve growth factor; fibroblast growth factor (e.g., aFGF and bFGF); epidermal growth factor (EGF); transforming growth factor (TGF, e.g., TGF-α and TGF-β); insulin-like growth factor binding proteins; erythropoietin; thrombopoietin; mucins;; growth hormone (e.g., human growth hormone); proinsulin, insulin A-chain insulin B-chain; parathyroid hormone; thyroid stimulating hormone; thyroxine; follicle stimulating hormone; calcitonin; atrial natriuretic peptides A, B or C; leutinizing hormone; glucagon; factor VIII; hemopoietic growth factor; tumor necrosis factor (e.g., TNF-α and TNF-β); enkephalinase; mullerian-inhibiting substance; gonadotropin-associated peptide tissue factor protein; inhibin; activin; vascular endothelial growth factor; receptors for hormones, growth factors, and other molecules described herein; protein A or D; rheumatoid factors; osteoinductive factors; an interferon, e.g., interferon-α,β,γ; colony stimulating factors (CSFs), e.g., M-CSF, GM-CSF, and G-CSF; interleukins (ILs), e.g., IL-1, IL-2, IL-3, IL-4, etc.; decay accelerating factor; immunoglobulin (constant or variable domains); and fragments of any of the above-listed polypeptides. In some embodiments, the target is associated with a disease, e.g., cancer.

### Sequences of Human Serum Proteins

The amino acid sequences of human serum proteins are well known and can be found in public sequence repositories, e.g., GenBank (National Center for Biotechnology Information, National Institutes of Health, Bethesda Md.). Further, in the human population, natural genetic variation can result in amino acid differences between serum proteins among individuals.

The following sequences are examples of at least some human serum protein amino acid sequences from particular individuals.

In many individuals, HSA has the amino acid sequence listed in SwissProt entry: P02768 and/or the following mature sequence:

| | |
|---|---|
| DAHKSEVAHRFKDLGEENFKALVLIAFAQYLQQCPFEDHVKLVNEVTEF | (SEQ ID NO:3) |
| AKTCVADESAENCDKSLHTLFGDKLCTVATLRETYGEMADCCAKQEPERNECFL | |
| QHKDDNPNLPRLVRPEVDVMCTAFHDNEETFLKKYLYEIARRHPYFYAPELLFF | |
| AKRYKAAFTECCQAADKAACLLPKLDELRDEGKASSAKQRLKCASLQKFGERA | |
| FKAWAVARLSQRFPKAEFAEVSKLVTDLTKVHTECCHGDLLECADDRADLAKY | |
| ICENQDSISSKLKECCEKPLLEKSHCIAEVENDEMPADLPSLAADFVESKDVCKN | |
| YAEAKDVFLGMFLYEYARRHPDYSVVLLLRLAKTYETTLEKCCAAADPHECYA | |
| KVFDEFKPLVEEPQNLIKQNCELFEQLGEYKFQNALLVRYTKKVPQVSTPTLVEV | |
| SRNLGKVGSKCCKHPEAKRMPCAEDYLSVVLNQLCVLHEKTPVSDRVTKCCTE | |
| SLVNRRPCFSALEVDETYVPKEFNAETFTFHADICTLSEKERQIKKQTALVELVK | |
| HKLPKATKEQLKAVMDDFAAFVEKCCKADDKETCFAEEGKKLVAASQAALGL. | |

Examples of human serum albumin variants include H27Q, H27Y, E106K, R122S, E378K, E400K, and E529K (numbered using the unprocessed sequence, wherein the initial D of SEQ ID NO: 1 corresponds to residue 25 of the unprocessed sequence).

Purified protein preparations of human serum albumin can be prepared by a variety of methods, including, for example, US Reissue 36,259 and U.S. Pat. No. 5,986,062.

In some cases, the serum albumin is a non-human serum albumin. For example, the amino acid sequence of one murine serum albumin is:

| | |
|---|---|
| MKWVTFLLLLFVSGSAFSRGVFRREAHKSEIAHRYNDLGEQHFKGLVLIA | (SEQ ID NO:4) |
| FSQYLQKCSYDEHAKLVQEVTDFAKTCVADESAANCDKSLHTLFGDKLCAIPNL | |
| RENYGELADCCTKQEPERNECFLQHKDDNPSLPPFERPEAEAMCTSFKENPTTFM | |
| GHYLHEVARRHPYFYAPELLYYAEQYNEILTQCCAEADKESCLTPKLDGVKEKA | |
| LVSSVRQRMKCSSMQKFGERAFKAWAVARLSQTFPNADFAEITKLATDLTKVN | |
| KECCHGDLLECADDRAELAKYMCENQATISSKLQTCCDKPLLKKAHCLSEVEHD | |
| TMPADLPAIAADFVEDQEVCKNYAEAKDVFLGTFLYEYSRRHPDYSVSLLLRLA | |
| KKYEATLEKCCAEANPPACYGTVLAEFQPLVEEPKNLVKTNCDLYEKLGEYGFQ | |
| NAILVRYTQKAPQVSTPTLVEAARNLGRVGTKCCTLPEDQRLPCVEDYLSAILNR | |
| VCLLHEKTPVSEHVTKCCSGSLVERRPCFSALTVDETYVPKEFKAETFTFHSDICT | |
| LPEKEKQIKKQTALAELVKHKPKATAEQLKTVMDDFAQFLDTCCKAADKDTCF | |
| STEGPNLVTRCKDALA | |

### Characterization of Binding Interactions

The binding properties of a ligand that binds a serum protein can be readily assessed using various assay formats. For example, the binding property of a ligand can be measured in solution by fluorescence anisotropy, which provides a convenient and accurate method of determining a dissociation constant (K_{D}) of a binding moiety for a serum albumin or for a particular molecular target. In one such procedure, a binding moiety described herein is labeled with fluorescein. The fluorescein-labeled binding moiety may then be mixed in wells of a multi-well assay plate with various concentrations of serum albumin or of the target. Fluorescence anisotropy measurements are then carried out using a fluorescence polarization plate reader.

ELISA. The binding interaction of a ligand for a target (or serum albumin) can also be analyzed using an ELISA assay. For example, the ligand is contacted to a microtitre plate whose bottom surface has been coated with the target, e.g., a limiting amount of the target. The molecule is contacted to the plate. The plate is washed with buffer to remove non-specifically bound molecules. Then the amount of the ligand bound to the plate is determined by probing the plate with an antibody specific to the ligand. The antibody can be linked to an enzyme such as alkaline phosphatase, which produces a calorimetric product when appropriate substrates are provided. In the case of a display library member, the antibody can recognize a region that is constant among all display library members, e.g., for a phage display library member, a major phage coat protein.

Homogeneous Assays. A binding interaction between a ligand and its target or serum albumin can be analyzed using a homogenous assay, i.e., after all components of the assay are added, additional fluid manipulations are not required. For example, fluorescence energy transfer (FET) can be used as a homogenous assay (see, for example, Lakowicz et al., U.S. Pat. No. 5,631,169; Stavrianopoulos, et al., U.S. Pat. No. 4,868,103). A fluorophore label on the first molecule (e.g., the molecule identified in the fraction) is selected such that its emitted fluorescent energy can be absorbed by a fluorescent label on a second molecule (e.g., the target) if the second molecule is in proximity to the first molecule. The fluorescent label on the second molecule fluoresces when it absorbs to the transferred energy. Since the efficiency of energy transfer between the labels is related to the distance separating the molecules, the spatial relationship between the molecules can be assessed. In a situation in which binding occurs between the molecules, the fluorescent emission of the ‘acceptor’ molecule label in the assay should be maximal. An FET binding event can be conveniently measured through standard fluorometric detection means well known in the art (e.g., using a fluorimeter). By titrating the amount of the first or second binding molecule, a binding curve can be generated to estimate the equilibrium binding constant.

Surface Plasmon Resonance (SPR). After a molecule is identified in a fraction, its binding interaction with a target can be analyzed using SPR. For example, after sequencing of a display library member present in a sample, and optionally verified, e.g., by ELISA, the displayed polypeptide can be produced in quantity and assayed for binding the target using SPR. SPR or real-time Biomolecular Interaction Analysis (BIA) detects biospecific interactions in real time, without labeling any of the interactants (e.g., BIAcore). Changes in the mass at the binding surface (indicative of a binding event) of the BIA chip result in alterations of the refractive index of light near the surface (the optical phenomenon of surface plasmon resonance (SPR)). The changes in the refractivity generate a detectable signal, which are measured as an indication of real-time reactions between biological molecules. Methods for using SPR are described, for example, in U.S. Pat. No. 5,641,640; Raether (1988) Surface Plasmons Springer Verlag; Sjolander, S. and Urbaniczky, C. (1991) Anal. Chem. 63:2338-2345; Szabo et al. (1995) Curr. Opin. Struct. Biol. 5:699-705.

Information from SPR can be used to provide an accurate and quantitative measure of the equilibrium dissociation constant (K_{d}), and kinetic parameters, including kₒₙ and k_{off}, for the binding of a biomolecule to a target. Such data can be used to compare different biomolecules. For example, proteins selected from a display library can be compared to identify individuals that have high affinity for the target or that have a slow k_{off}. This information can also be used to develop structure-activity relationship (SAR) if the biomolecules are related. For example, if the proteins are all mutated variants of a single parental antibody or a set of known parental antibodies, variant amino acids at given positions can be identified that correlate with particular binding parameters, e.g., high affinity and slow k_{off}.

Additional methods for measuring binding affinities include fluorescence polarization (FP) (see, e.g., U.S. Pat. No. 5,800,989), nuclear magnetic resonance (NMR), and binding titrations (e.g., using fluorescence energy transfer).

Other solution measures for studying binding properties include fluorescence resonance energy transfer (FRET) and NMR.

### Characterization of In Vivo Half-Life

Ligands can also be characterized to determine their in vivo half life or efficacy. One exemplary method for measuring in vivo half life is as follows:

The ligand is first labeled. For example, the ligand can be labeled directly, e.g., on tyrosine using I¹²⁵ (e.g., iodo-gen or iodo-beads) or the ligand can be coupled to a chelator to prepare a Tc or Indium chelate, e.g., with ^{99m}Tc or ¹¹¹In. The labeled ligands are injected into mice. The mice are sacrificed at different time points and serum collected from each time point. The amount of label in each sample is counted to generate a curve for ligand concentration vs. time.

Other animals, such as another rodent (e.g., a rat), can also be used. It may be useful to verify that the ligand being tested also binds to the serum albumin of the animal as well as to HSA before testing. It may even be useful to screen for a ligand that does not bind to serum albumin in a species specific manner.

Ligands that have a half-life of at least 30, 40, 60, 80, 120, 240 minutes, or greater than 5, 8, 12, 20, 24, or 36 hours, or greater than 2 or 4 days in a mouse, rat, chimp, and/or human individual can be particularly useful.

### Ligand Production

Standard recombinant nucleic acid methods can be used to express a protein ligand that interacts with a target and binds to serum albumin. In one embodiment, a nucleic acid sequence encoding the protein ligand is cloned into a nucleic acid expression vector, e.g., with appropriate signal and processing sequences and regulatory sequences for transcription and translation. In another embodiment, particularly for peptide ligands, the protein can be synthesized using automated organic synthetic methods. Synthetic methods for producing proteins are described, for example in Methods in Enzymology, Volume 289: Solid-Phase Peptide Synthesis by Gregg B. Fields (Editor), Sidney P. Colowick, Melvin I. Simon (Editor), Academic Press; (November 15, 1997) ISBN:0121821900.

The expression vector for expressing the protein ligand can include, in addition to the segment encoding the protein ligand or fragment thereof, regulatory sequences, including for example, a promoter, operably linked to the nucleic acid(s) of interest. Large numbers of suitable vectors and promoters are known to those of skill in the art and are commercially available for generating the recombinant constructs of the present invention. The following vectors are provided by way of example. Bacterial: pBs, phagescript, PsiX174, pBluescript SK, pBs KS, pNH8a, pNH16a, pNH18a, pNH46a (Stratagene); pTrc99A, pKK223-3, pKK233-3, pDR540, and pRIT5 (Pharmacia). Eukaryotic: pWLneo, pSV2cat, pOG44, PXTI, pSG (Stratagene) pSVK3, pBPV, pMSG, and pSVL (Pharmacia).

Methods well known to those skilled in the art can be used to construct vectors containing a polynucleotide of the invention and appropriate transcriptional/translational control signals. These methods include in vitro recombinant DNA techniques, synthetic techniques and in vivo recombination/genetic recombination. See, for example, the techniques described in Sambrook & Russell, Molecular Cloning: A Laboratory Manual, 3rd Edition, Cold Spring Harbor Laboratory, N.Y. (2001) and Ausubel et al., Current Protocols in Molecular Biology (Greene Publishing Associates and Wiley Interscience, N.Y. (1989). Promoter regions can be selected from any desired gene using CAT (chloramphenicol transferase) vectors or other vectors with selectable markers. Two appropriate vectors are pKK232-8 and pCM7. Particular named bacterial promoters include lacI, lacZ, T3, T7, gpt, lambda P, and trc. Eukaryotic promoters include CMV immediate early, HSV thymidine kinase, early and late SV40, LTRs from retrovirus, mouse metallothionein-I, and various art-known tissue specific promoters.

Exemplary prokaryotic hosts for transformation include E. coli, Bacillus subtilis, Salmonella typhimurium and various species within the genera Pseudomonas, Streptomyces, and Staphylococcus, although others may also be employed as a matter of choice. Exemplary eukaryotic hosts include yeast, mammalian cells (e.g., HeLa cells, CV-1 cell, COS cells) and insect cells (e.g,.Sf9 cells). The host of the present invention may also be a yeast or other fungi. In yeast, a number of vectors containing constitutive or inducible promoters may be used. For a review see, Current Protocols in Molecular Biology, Vol. 2, Ed. Ausubel et al., Greene Publish. Assoc. & Wiley Interscience, Ch. 13 (1988); Grant et al., Expression and Secretion Vectors for Yeast, in Methods in Enzymology, Ed. Wu & Grossman, Acad. Press, N.Y. 153:516-544 (1987); Glover, DNA Cloning, Vol. II, IRL Press, Wash., D.C., Ch. 3 (1986); Bitter, Heterologous Gene Expression in Yeast, in Methods in Enzymology, Eds. Berger & Kimmel, Acad. Press, N.Y. 152:673-684 (1987); and The Molecular Biology of the Yeast Saccharomyces, Eds. Strathem et al., Cold Spring Harbor Press, Vols. I and 11 (1982). Potentially suitable yeast strains include Saccharomyces cerevisiae, Schizosaccharomyces pombe, Kluyveromyces strains, Candida, or any yeast strain capable of expressing heterologous proteins.

Examples of mammalian expression systems include the COS-7 lines of monkey kidney fibroblasts, described by Gluzman, Cell 23:175 (1981), and other cell lines capable of expressing a compatible vector, for example, the C127, 3T3, CHO, HeLa and BHK cell lines. Mammalian expression vectors will comprise an origin of replication, a suitable promoter and also any necessary ribosome-binding sites, polyadenylation site, splice donor and acceptor sites, transcriptional termination sequences, and 5′ flanking nontranscribed sequences. Mammalian host cells include, for example, monkey COS cells, Chinese Hamster Ovary (CHO) cells, human kidney 293 cells, human epidermal A431 cells, human Colo2O5 cells, 3T3 cells, CV-1 cells, other transformed primate cell lines, normal diploid cells, cell strains derived from in vitro culture of primary tissue, primary explants, HeLa cells, mouse L cells, BHK, HL-60, U937, HaK or Jurkat cells.

### Treatments

Protein ligands that bind to a target and to serum albumin, e.g., ligands identified by the method described herein and/or detailed herein have therapeutic and prophylactic utilities. For example, these ligands can be administered to a subject, e.g., in vivo, to treat, prevent, and/or diagnose a variety of disorders, such as cancers.

As used herein, the term “treat” or “treatment” is defined as the application or administration of a target-specific ligand, alone or in combination with, a second agent to a subject, e.g., a patient, or application or administration of the agent to an isolated tissue or cell, e.g., cell line, from a subject, e.g., a patient, who has a disorder (e.g., a disorder as described herein), a symptom of a disorder or a predisposition toward a disorder, with the purpose to cure, heal, alleviate, relieve, alter, remedy, ameliorate, improve or affect the disorder, the symptoms of the disorder or the predisposition toward the disorder. Treating a cell refers to the inhibition, ablation, killing of a cell in vitro or in vivo, or otherwise reducing capacity of a cell, e.g., an aberrant cell, to mediate a disorder, e.g., a disorder as described herein (e.g., a cancerous disorder). In one embodiment, “treating a cell” refers to a reduction in the activity and/or proliferation of a cell, e.g., a hyperproliferative cell. Such reduction does not necessarily indicate a total elimination of the cell, but a reduction, e.g., a statistically significant reduction, in the activity or the number of the cell.

As used herein, an amount of a target-specific ligand effective to treat a disorder, or a “therapeutically effective amount” refers to an amount of the ligand which is effective, upon single or multiple dose administration to a subject, in treating a cell, e.g., a cancer cell (e.g., a target-expressing cancer cell), or in prolonging curing, alleviating, relieving or improving a subject with a disorder as described herein beyond that expected in the absence of such treatment. As used herein, “inhibiting the growth” of the neoplasm refers to slowing, interrupting, arresting or stopping its growth and metastases and does not necessarily indicate a total elimination of the neoplastic growth.

As used herein, an amount of a target-specific ligand effective to prevent a disorder, or a “a prophylactically effective amount” of the ligand refers to an amount of a target-specific ligand, e.g., a target-specific ligand described herein, which is effective, upon single- or multiple-dose administration to the subject, in preventing or delaying the occurrence of the onset or recurrence of a disorder, e.g., a cancer.

The terms “induce”, “inhibit”, “potentiate”, “elevate”, “increase”, “decrease” or the like, e.g., which denote quantitative differences between two states, refer to a difference, e.g., a statistically significant difference, between the two states. For example, “an amount effective to inhibit the proliferation of the target-expressing cells” means that the rate of growth of the cells will be different, e.g., statistically significantly different, from the untreated cells.

As used herein, the term “subject” is intended to include human and non-human animals. Preferred human animals include a human patient having a disorder characterized by abnormal cell proliferation or cell differentiation. The term “non-human animals” includes all vertebrates, e.g., non-mammals (such as chickens, amphibians, reptiles) and non-human mammals, such as non-human primates, sheep, dog, cow, pig, etc.

In one embodiment, the subject is a human subject. Alternatively, the subject can be a mammal expressing a target molecule with which a target-specific ligand cross-reacts. A target-specific ligand can be administered to a human subject for therapeutic purposes (discussed further below). Moreover, a target-specific ligand can be administered to a non-human mammal expressing the target or homlog thereof to which the ligand binds (e.g., a primate, pig or mouse) for veterinary purposes or as an animal model of human disease. Regarding the latter, such animal models may be useful for evaluating the therapeutic efficacy of the ligand (e.g., testing of dosages and time courses of administration).

In one embodiment, the invention provides a method of treating (e.g., reducing growth, reducing proliferation, ablating or killing) a cell (e.g., a non-cancerous cell, e.g., a normal, benign or hyperplastic cell, or a cancerous cell, e.g., a malignant cell, e.g., cell found in a solid tumor, a soft tissue tumor, or a metastatic lesion (e.g., a cell found in renal, urothelial, colonic, rectal, pulmonary, breast or hepatic, cancers and/or metastasis))s. Methods of the invention include the steps of contacting the cell with a target-specific ligand, e.g., a target-specific ligand described herein, in an amount sufficient to treat the cell.

The subject method can be used on cells in culture, e.g. in vitro or ex vivo. For example, cancerous or metastatic cells (e.g., renal, urothelial, colon, rectal, lung, breast, ovarian, prostatic, or liver cancerous or metastatic cells) can be cultured in vitro in culture medium and the contacting step can be effected by adding a target-specific ligand to the culture medium. The method can be performed on cells (e.g., cancerous or metastatic cells) present in a subject, as part of an in vivo (e.g., therapeutic or prophylactic) protocol. For in vivo embodiments, the contacting step is effected in a subject and includes administering a target-specific ligand to the subject under conditions effective to permit both binding of the ligand to the cell and the treating, e.g., the killing or ablating of the cell.

The method can be used to treat a cancer. As used herein, the terms “cancer”, “hyperproliferative”, “malignant”, and “neoplastic” are used interchangeably, and refer to those cells an abnormal state or condition characterized by rapid proliferation or neoplasm. The terms include all types of cancerous growths or oncogenic processes, metastatic tissues or malignantly transformed cells, tissues, or organs, irrespective of histopathologic type or stage of invasiveness. “Pathologic hyperproliferative” cells occur in disease states characterized by malignant tumor growth.

The common medical meaning of the term “neoplasia” refers to “new cell growth” that results as a loss of responsiveness to normal growth controls, e.g. to neoplastic cell growth. A “hyperplasia” refers to cells undergoing an abnormally high rate of growth. However, as used herein, the terms neoplasia and hyperplasia can be used interchangeably, as their context will reveal, referring generally to cells experiencing abnormal cell growth rates. Neoplasias and hyperplasias include “tumors,” which may be benign, premalignant or malignant.

Examples of cancerous disorders include, but are not limited to, solid tumors, soft tissue tumors, and metastatic lesions. Examples of solid tumors include malignancies, e.g., sarcomas, adenocarcinomas, and carcinomas, of the various organ systems, such as those affecting lung, breast, lymphoid, gastrointestinal (e.g., colon), and genitourinary tract (e.g., renal, urothelial cells), pharynx, prostate, ovary as well as adenocarcinomas which include malignancies such as most colon cancers, rectal cancer, renal-cell carcinoma, liver cancer, non-small cell carcinoma of the lung, cancer of the small intestine and so forth. Metastatic lesions of the aforementioned cancers can also be treated or prevented using the methods and compositions of the invention.

The subject method can also be used to inhibit the proliferation of hyperplastic/neoplastic cells of hematopoietic origin, e.g., arising from myeloid, lymphoid or erythroid lineages, or precursor cells thereof.

Methods of administering a target-specific ligand are described in “Pharmaceutical Compositions”. Suitable dosages of the molecules used will depend on the age and weight of the subject and the particular drug used. The ligands can be used as competitive agents to inhibit, reduce an undesirable interaction, e.g., between a natural or pathological agent and the target.

In one embodiment, the target-specific ligands are used to kill or ablate cancerous cells and normal, benign hyperplastic, and cancerous cells in vivo. The ligands can be used by themselves or conjugated to an agent, e.g., a cytotoxic drug, radioisotope. This method includes: administering the ligand alone or attached to a cytotoxic drug, to a subject requiring such treatment.

The terms “cytotoxic agent” and “cytostatic agent” and “anti-tumor agent” are used interchangeably herein and refer to agents that have the property of inhibiting the growth or proliferation (e.g., a cytostatic agent), or inducing the killing, of hyperproliferative cells, e.g., an aberrant cancer cell. In cancer therapeutic embodiment, the term “cytotoxic agent” is used interchangeably with the terms “anti-cancer” or “antitumor” to mean an agent, which inhibits the development or progression of a neoplasm, particularly a solid tumor, a soft tissue tumor, or a metastatic lesion.

Nonlimiting examples of anti-cancer agents include, e.g., antimicrotubule agents, topoisomerase inhibitors, antimetabolites, mitotic inhibitors, alkylating agents, intercalating agents, agents capable of interfering with a signal transduction pathway, agents that promote apoptosis, radiation, and antibodies against other tumor-associated antigens (including naked antibodies, immunotoxins and radioconjugates). Examples of the particular classes of anti-cancer agents are provided in detail as follows: antitubulin/antimicrotubule, e.g., paclitaxel, vincristine, vinblastine, vindesine, vinorelbin, taxotere; topoisomerase I inhibitors, e.g., topotecan, camptothecin, doxorubicin, etoposide, mitoxantrone, daunorubicin, idarubicin, teniposide, amsacrine, epirubicin, merbarone, piroxantrone hydrochloride; antimetabolites, e.g., 5-fluorouracil (5-FU), methotrexate, 6-mercaptopurine, 6-thioguanine, fludarabine phosphate, cytarabine/Ara-C, trimetrexate, gemcitabine, acivicin, alanosine, pyrazofurin, N-Phosphoracetyl-L-Asparate=PALA, pentostatin, 5-azacitidine, 5-Aza 2′-deoxycytidine, ara-A, cladribine, 5 -fluorouridine, FUDR, tiazofurin, N-[5-[N-(3,4-dihydro-2-methyl-4-oxoquinazolin-6-ylmethyl)-N-methylamino]-2-thenoyl]-L-glutamic acid; alkylating agents, e.g., cisplatin, carboplatin, mitomycin C, BCNU=Carmustine, melphalan, thiotepa, busulfan, chlorambucil, plicamycin, dacarbazine, ifosfamide phosphate, cyclophosphamide, nitrogen mustard, uracil mustard, pipobroman, 4-ipomeanol; agents acting via other mechanisms of action, e.g., dihydrolenperone, spiromustine, and desipeptide; biological response modifiers, e.g., to enhance anti-tumor responses, such as interferon; apoptotic agents, such as actinomycin D; and anti-hormones, for example anti-estrogens such as tamoxifen or, for example antiandrogens such as 4′-cyano-3-(4-fluorophenylsulphonyl)-2-hydroxy-2-methyl-3′-(trigluoromethyl) propionanilide.

Some target-specific ligands (e.g., modified with a cytotoxin) can selectively kill or ablate cells in cancerous tissue (including the cancerous cells themselves) and/or cells in the vicinity

The ligands may be used to deliver a variety of cytotoxic drugs including therapeutic drugs, a compound emitting radiation, molecules of plants, fungal, or bacterial origin, biological proteins, and mixtures thereof. The cytotoxic drugs can be intracellularly acting cytotoxic drugs, such as short-range radiation emitters, including, for example, short-range, high-energy a-emitters, as described herein.

Enzymatically active toxins and fragments thereof are exemplified by diphtheria toxin A fragment, nonbinding active fragments of diphtheria toxin, exotoxin A (from Pseudomonas aeruginosa), ricin A chain, abrin A chain, modeccin A chain, α-sacrin, certain Aleurites fordii proteins, certain Dianthin proteins, Phytolacca americana proteins (PAP, PAPII and PAP-S), Morodica charantia inhibitor, curcin, crotin, Saponaria officinalis inhibitor, gelonin, mitogillin, restrictocin, phenomycin, and enomycin. Procedures for preparing enzymatically active polypeptides of the immunotoxins are described in W084/03508 and W085/03508, which are hereby incorporated by reference. Examples of cytotoxic moieties that can be conjugated to the antibodies include adriamycin, chlorambucil, daunomycin, methotrexate, neocarzinostatin, and platinum.

In the case of polypeptide toxins, recombinant nucleic acid techniques can be used to construct a nucleic acid that encodes the ligand (or a polypeptide component thereof) and the cytotoxin (or a polypeptide component thereof) as translational fusions. The recombinant nucleic acid is then expressed, e.g., in cells and the encoded fusion polypeptide isolated.

Procedures for conjugating protein ligands (e.g., antibodies) with the cytotoxic agents have been previously described. Procedures for conjugating chlorambucil with antibodies are described by Flechner (1973) European Journal of Cancer, 9:741-745; Ghose et al. (1972) British Medical Journal, 3:495-499; and Szekerke, et al. (1972) Neoplasma, 19:211-215, which are hereby incorporated by reference. Procedures for conjugating daunomycin and adriamycin to antibodies are described by Hurwitz, E. et al. (1975) Cancer Research, 35:1175-1181 and Arnon et al. (1982) Cancer Surveys, 1:429-449, which are hereby incorporated by reference. Procedures for preparing antibody-ricin conjugates are described in U.S. Pat. No. 4,414,148 and by Osawa, T., et al. (1982) Cancer Surveys, 1:373-388 and the references cited therein, which are hereby incorporated by reference. Coupling procedures as also described in EP 86309516.2, which is hereby incorporated by reference.

To kill or ablate normal, benign hyperplastic, or cancerous cells, a first protein ligand is conjugated with a prodrug which is activated only when in close proximity with a prodrug activator. The prodrug activator is conjugated with a second protein ligand, preferably one which binds to a non-competing site on the target molecule. Whether two protein ligands bind to competing or non-competing binding sites can be determined by conventional competitive binding assays. Drug-prodrug pairs suitable for use in the practice of the present invention are described in Blakely et al., (1996) Cancer Research, 56:3287-3292.

Alternatively, a target-specific ligand can be coupled to high energy radiation emitters, for example, a radioisotope, such as ¹³¹I, a γ-emitter, which, when localized at the tumor site, results in a killing of several cell diameters. See, e.g., S. E. Order, “Analysis, Results, and Future Prospective of the Therapeutic Use of Radiolabeled Antibody in Cancer Therapy”, Monoclonal Antibodies for Cancer Detection and Therapy, R. W. Baldwin et al. (eds.), pp 303-316 (Academic Press 1985). Other suitable radioisotopes include α-emitters, such as ²¹²Bi, ²¹³Bi, and ²¹¹At, and β-emitters, such as 186Re and ⁹⁰Y. Moreover, Lu¹¹⁷ may also be used as both an imaging and cytotoxic agent.

Radioimmunotherapy (RIT) using antibodies labeled with ¹³¹I , ⁹⁰Y, and ¹⁷⁷Lu is under intense clinical investigation. There are significant differences in the physical characteristics of these three nuclides and as a result, the choice of radionuclide is very critical in order to deliver maximum radiation dose to the tumor. The higher beta energy particles of ⁹⁰Y may be good for bulky tumors. The relatively low energy beta particles of 13 1 are ideal, but in vivo dehalogenation of radioiodinated molecules is a major disadvantage for internalizing antibody. In contrast, ¹⁷⁷Lu has low energy beta particle with only 0.2-0.3 mm range and delivers much lower radiation dose to bone marrow compared to ⁹⁰Y. In addition, due to longer physical half-life (compared to ⁹⁰Y), the tumor residence times are higher. As a result, higher activities (more mCi amounts) of ¹⁷⁷Lu labeled agents can be administered with comparatively less radiation dose to marrow. There have been several clinical studies investigating the use of 1⁷⁷Lu labeled antibodies in the treatment of various cancers. (Mulligan T et al. (1995) Clin Cancer Res. 1: 1447-1454; Meredith R F, et al. (1996) J Nucl Med 37:1491-1496; Alvarez R D, et al. (1997) Gynecologic Oncology 65: 94-101).

The target-specific ligands can be used directly in vivo to eliminate antigen-expressing cells via natural complement-dependent cytotoxicity (CDC) or antibody-dependent cellular cytotoxicity (ADCC). Certain protein ligands can include complement binding effector domain, such as the Fc portions from IgG1, -2, or -3 or corresponding portions of IgM which bind complement or peptides which can bind to complement proteins. In one embodiment, a population of target cells is ex vivo treated with a target-specific ligand and appropriate effector cells. The treatment can be supplemented by the addition of complement or serum containing complement. Further, phagocytosis of target cells coated with a protein ligand can be improved by binding of complement proteins. In another embodiment target, cells coated with the protein ligand which includes a complement binding effector domain are lysed by complement.

Also encompassed by the present invention is a method of killing or ablating which involves using the a target-specific ligand for prophylaxis. For example, these materials can be used to prevent or delay development or progression of cancers.

Use of the therapeutic methods of the present invention to treat cancers has a number of benefits. Since the protein ligands specifically recognize a target protein, other tissue is spared and high levels of the agent are delivered directly to the site where therapy is required. Treatment in accordance with the present invention can be effectively monitored with clinical parameters. Alternatively, these parameters can be used to indicate when such treatment should be employed.

Target-specific ligands can be administered in combination with one or more of the existing modalities for treating cancers, including, but not limited to: surgery; radiation therapy, and chemotherapy.

### Pharmaceutical Compositions

In another aspect, the present invention provides compositions, e.g., pharmaceutically acceptable compositions, which include a target-specific ligand (e.g., a ligand that interacts with (e.g., specifically binds to) a target (e.g., a target molecule, target cell, or target tissue) and that binds to a serum albumin, or a polypeptide identified as binding to a target and to a serum albumin (as described herein) formulated together with a pharmaceutically acceptable carrier. As used herein, “pharmaceutical compositions” encompass labeled ligands, e.g., for in vivo imaging as well as therapeutic compositions.

As used herein, “pharmaceutically acceptable carrier” includes any and all solvents, dispersion media, coatings, antibacterial and antifungal agents, isotonic and absorption delaying agents, and the like that are physiologically compatible. Preferably, the carrier is suitable for intravenous, intramuscular, subcutaneous, parenteral, spinal or epidermal administration (e.g., by injection or infusion). Depending on the route of administration, the active compound, i.e., protein ligand may be coated in a material to protect the compound from the action of acids and other natural conditions that may inactivate the compound.

A “pharmaceutically acceptable salt” refers to a salt that retains the desired biological activity of the parent compound and does not impart any undesired toxicological effects (see e.g., Berge, S. M., et al. (1977) J. Pharm. Sci. 66:1-19). Examples of such salts include acid addition salts and base addition salts. Acid addition salts include those derived from nontoxic inorganic acids, such as hydrochloric, nitric, phosphoric, sulfuric, hydrobromic, hydroiodic, phosphorous and the like, as well as from nontoxic organic acids such as aliphatic mono- and dicarboxylic acids, phenyl-substituted alkanoic acids, hydroxy alkanoic acids, aromatic acids, aliphatic and aromatic sulfonic acids and the like. Base addition salts include those derived from alkaline earth metals, such as sodium, potassium, magnesium, calcium and the like, as well as from nontoxic organic amines, such as N,N′-dibenzylethylenediamine, N-methylglucamine, chloroprocaine, choline, diethanolamine, ethylenediamine, procaine and the like.

The compositions of this invention may be in a variety of forms. These include, for example, liquid, semi-solid and solid dosage forms, such as liquid solutions (e.g., injectable and infusible solutions), dispersions or suspensions, tablets, pills, powders, liposomes and suppositories. The preferred form depends on the intended mode of administration and therapeutic application. Typical preferred compositions are in the form of injectable or infuisible solutions, such as compositions similar to those used for administration of humans with antibodies. The preferred mode of administration is parenteral (e.g., intravenous, subcutaneous, intraperitoneal, intramuscular). In a preferred embodiment, the ligand is administered by intravenous infusion or injection.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrastemal injection and infusion.

Pharmaceutical compositions typically must be sterile and stable under the conditions of manufacture and storage. A pharmaceutical composition can also be tested to insure it meets regulatory and industry standards for administration. For example, endotoxin levels in the preparation can be tested using the Limulus amebocyte lysate assay (e.g., using the kit from Bio Whittaker lot #7L3790, sensitivity 0.125 EU/mL) according to the USP 24/NF 19 methods. Sterility of pharmaceutical compositions can be determined using thioglycollate medium according to the USP 24/NF 19 methods. For example, the preparation is used to inoculate the thioglycollate medium and incubated at 35° C. for 14 or more days. The medium is inspected periodically to detect growth of a microorganism.

The composition can be formulated as a solution, microemulsion, dispersion, liposome, or other ordered structure suitable to high drug concentration. Sterile injectable solutions can be prepared by incorporating the active compound (i.e., the ligand) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying that yields a powder of the active ingredient plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

The target-specific ligands can be administered by a variety of methods known in the art, although for many applications, the preferred route/mode of administration is intravenous injection or infusion. For example, for therapeutic applications, the ligand can be administered by intravenous infusion at a rate of less than 30, 20, 10, 5, or 1 mg/min to reach a dose of about 1 to 100 mg/m² or 7 to 25 mg/m². The route and/or mode of administration will vary depending upon the desired results. In certain embodiments, the active compound may be prepared with a carrier that will protect the compound against rapid release, such as a controlled release formulation, including implants, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known. See, e.g., Sustained and Controlled Release Drug Delivery Systems, J. R. Robinson, ed., Marcel Dekker, Inc., New York, 1978.

Pharmaceutical compositions can be administered with medical devices known in the art. For example, in a preferred embodiment, a pharmaceutical composition can be administered with a needleless hypodermic injection device, such as the devices disclosed in U.S. Pat. Nos. 5,399,163, 5,383,851, 5,312,335, 5,064,413, 4,941,880, 4,790,824, or 4,596,556. Examples of well-known implants and modules useful in the present invention include: U.S. Pat. No. 4,487,603, which discloses an implantable micro-infusion pump for dispensing medication at a controlled rate; U.S. Pat. No. 4,486,194, which discloses a therapeutic device for administering medicaments through the skin; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments; and U.S. Pat. No. 4,475,196, which discloses an osmotic drug delivery system. Of course, many other such implants, delivery systems, and modules are also known.

In certain embodiments, the compounds described herein can be formulated to ensure proper distribution in vivo. For example, the blood-brain barrier (BBB) excludes many highly hydrophilic compounds. To ensure that the therapeutic compounds cross the BBB (if desired), they can be formulated, for example, in liposomes. For methods of manufacturing liposomes, see, e.g., U.S. Pat. Nos. 4,522,811; 5,374,548; and 5,399,331. The liposomes may comprise one or more moieties which are selectively transported into specific cells or organs, thus enhance targeted drug delivery (see, e.g., Ranade (1989) J. Clin. Pharmacol. 29:685).

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier. The specification for the dosage unit may be dictated by and directly dependent on (a) the unique characteristics of the active compound and the particular therapeutic effect to be achieved, and (b) the limitations inherent in the art of compounding such an active compound for the treatment of sensitivity in individuals.

An exemplary, non-limiting range for a therapeutically or prophylactically effective amount of an antibody is 0.1-20 mg/kg, more preferably 1-10 mg/kg. The target-specific ligand can be administered by intravenous infusion at a rate of less than 30, 20, 10, 5, or 1 mg/min to reach a dose of about 1 to 100 mg/m² or about 5 to 30 mg/m². For ligands smaller in molecular weight than an antibody, appropriate amounts can be proportionally less, e.g., about 0.01-5 mg/kg or 0.005-1 mg/kg. It is to be noted that dosage values may vary with the type and severity of the condition to be alleviated. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition.

The pharmaceutical compositions may include a “therapeutically effective amount” or a “prophylactically effective amount” of a target-specific ligand. A “therapeutically effective amount” refers to an amount effective, at dosages and for periods of time necessary, to achieve the desired therapeutic result. A therapeutically effective amount of the composition may vary according to factors such as the disease state, age, sex, and weight of the individual, and the ability of the protein ligand to elicit a desired response in the individual. A therapeutically effective amount is also one in which any toxic or detrimental effects of the composition is outweighed by the therapeutically beneficial effects. A “therapeutically effective dosage” preferably inhibits a measurable parameter, e.g., tumor growth rate by at least about 20%, more preferably by at least about 40%, even more preferably by at least about 60%, and still more preferably by at least about 80% relative to untreated subjects. The ability of a compound to inhibit a measurable parameter, e.g., cancer, can be evaluated in an animal model system predictive of efficacy in human tumors. Alternatively, this property of a composition can be evaluated by examining the ability of the compound to inhibit, such inhibition in vitro by assays known to the skilled practitioner.

A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

Also within the scope of the invention are kits comprising the protein ligand that binds to a target molecule and to a serum albumin and instructions for use, e.g., treatment, prophylactic, or diagnostic use. In one embodiment, the instructions for diagnostic applications include the use of the ligand to detect a target expressing cell, in vitro, e.g., in a sample, e.g., a biopsy or cells from a patient having a cancer or neoplastic disorder, or in vivo. In another embodiment, the instructions for therapeutic applications include suggested dosages and/or modes of administration in a patient with a cancer or neoplastic disorder. The kit can further contain a least one additional reagent, such as a diagnostic or therapeutic agent, e.g., a diagnostic or therapeutic agent as described herein, and/or one or more additional target-specific ligands, formulated as appropriate, in one or more separate pharmaceutical preparations.

### Diagnostic Uses

Protein ligands that bind to a specific target molecule and to a serum albumin also have in vitro and in vivo diagnostic utilities.

In one aspect, the present invention provides a diagnostic method for detecting the presence of a target-expressing cell in vivo (e.g., in vivo imaging in a subject).

The method includes: (i) administering a target-specific ligand to a subject; and (iii) detecting formation of a complex between the ligand, and the subject. The detecting can include determining location or time of formation of the complex.

The ligand can be directly or indirectly labeled with a detectable substance to facilitate detection of the bound or unbound antibody. Suitable detectable substances include various enzymes, prosthetic groups, fluorescent materials, luminescent materials and radioactive materials.

In vivo Imaging. In still another embodiment, the invention provides a method for detecting the presence of a target-expressing cells or tissues in vivo. The method includes (i) administering to a subject (e.g., a patient having a cancer or neoplastic disorder) a target-specific ligand that binds to a serum albumin, the ligand being conjugated to a detectable marker; (ii) exposing the subject to a means for detecting said detectable marker. For example, the subject is imaged, e.g., by NMR or other tomographic means.

Examples of labels useful for diagnostic imaging in accordance with the present invention include radiolabels such as ¹³¹I, ¹¹¹In, ¹²³I, ^{99m}Tc, ³²P, ¹²⁵I, ³H, ¹⁴C, and ¹⁸⁸Rh, fluorescent labels such as fluorescein and rhodamine, nuclear magnetic resonance active labels, positron emitting isotopes detectable by a positron emission tomography (“PET”) scanner, chemiluminescers such as luciferin, and enzymatic markers such as peroxidase or phosphatase. Short-range radiation emitters, such as isotopes detectable by short-range detector probes can also be employed. The protein ligand can be labeled with such reagents using known techniques. For example, see Wensel and Meares (1983) Radioimmunoimaging and Radioimmunotherapy, Elsevier, N.Y. for techniques relating to the radiolabeling of antibodies and D. Colcher et al. (1986) Meth. Enzymol. 121: 802-816.

A radiolabeled ligand of this invention can also be used for in vitro diagnostic tests. The specific activity of a isotopically-labeled ligand depends upon the half-life, the isotopic purity of the radioactive label, and how the label is incorporated into the antibody.

Procedures for labeling polypeptides with the radioactive isotopes (such as ¹⁴C, ³H, ³⁵S, ¹²⁵I, ³²P, ¹³¹I) are generally known. For example, tritium labeling procedures are described in U.S. Pat. No. 4,302,438. lodinating, tritium labeling, and ³⁵S labeling procedures, e.g., as adapted for murine monoclonal antibodies, are described, e.g., by Goding, J. W. (Monoclonal antibodies: principles and practice: production and application of monoclonal antibodies in cell biology, biochemistry, and immunology 2nd ed. London; Orlando: Academic Press, 1986. pp 124-126) and the references cited therein. Other procedures for iodinating polypeptides, such as antibodies, are described by Hunter and Greenwood (1962) Nature 144:945, David et al. (1974) Biochemistry 13:1014-1021, and U.S. Pat. Nos. 3,867,517 and 4,376,110. Radiolabeling elements which are useful in imaging include ¹²³I, ¹³¹I, ¹¹¹In, and ^{99m}Tc, for example. Procedures for iodinating antibodies are described by Greenwood, F. et al. (1963) Biochem. J. 89:114-123; Marchalonis, J. (1969) Biochem. J. 113:299-305; and Morrison, M. et al. (1971) Immunochemistry 289-297. Procedures for ^{99m}Tc-labeling are described by Rhodes, B. et al. in Burchiel, S. et al. (eds.), Tumor Imaging: The Radioimmunochemical Detection of Cancer, New York: Masson 111-123 (1982) and the references cited therein. Procedures suitable for ¹¹¹In-labeling antibodies are described by Hnatowich, D. J. et al. (1983) J. Immul. Methods, 65:147-157, Hnatowich, D. et al. (1984) J. Applied Radiation, 35:554-557, and Buckley, R. G. et al. (1984) F.E.B.S. 166:202-204.

In the case of a radiolabeled ligand, the ligand is administered to the patient, is localized to the tumor bearing the antigen with which the ligand reacts, and is detected or “imaged” in vivo using known techniques such as radionuclear scanning using e.g., a gamma camera or emission tomography. See e.g., A. R. Bradwell et al., “Developments in Antibody Imaging”, Monoclonal Antibodies for Cancer Detection and Therapy, R. W. Baldwin et al., (eds.), pp 65-85 (Academic Press 1985). Alternatively, a positron emission transaxial tomography scanner, such as designated Pet VI located at Brookhaven National Laboratory, can be used where the radiolabel emits positrons (e.g., ¹¹C, ¹⁸F, ¹⁵O and ¹³N).

MRI Contrast Agents. Magnetic Resonance Imaging (MRI) uses NMR to visualize internal features of living subject, and is useful for prognosis, diagnosis, treatment, and surgery. MRI can be used without radioactive tracer compounds for obvious benefit. Some MRI techniques are summarized in EP-A-0 502 814. Generally, the differences related to relaxation time constants T1 and T2 of water protons in different environments is used to generate an image. However, these differences can be insufficient to provide sharp high resolution images.

The differences in these relaxation time constants can be enhanced by contrast agents. Examples of such contrast agents include a number of magnetic agents paramagnetic agents (which primarily alter T1) and ferromagnetic or superparamagnetic (which primarily alter T2 response). Chelates (e.g., EDTA, DTPA and NTA chelates) can be used to attach (and reduce toxicity) of some paramagnetic substances (e.g., . Fe⁺³, Mn⁺², Gd⁺³). Other agents can be in the form of particles, e.g., less than 10 μm to about 10 nM in diameter). Particles can have ferromagnetic, antiferromagnetic or superparamagnetic properties. Particles can include, e.g., magnetite (Fe₃O₄), γ-Fe₂O₃, ferrites, and other magnetic mineral compounds of transition elements. Magnetic particles may include: one or more magnetic crystals with and without nonmagnetic material. The nonmagnetic material can include synthetic or natural polymers (such as sepharose, dextran, dextrin, starch and the like The target-specific ligands can also be labeled with an indicating group containing of the NMR-active ¹⁹F atom, or a plurality of such atoms inasmuch as (i) substantially all of naturally abundant fluorine atoms are the ¹⁹F isotope and, thus, substantially all fluorine-containing compounds are NMR-active; (ii) many chemically active polyfluorinated compounds such as trifluoracetic anhydride are commercially available at relatively low cost, and (iii) many fluorinated compounds have been found medically acceptable for use in humans such as the perfluorinated polyethers utilized to carry oxygen as hemoglobin replacements. After permitting such time for incubation, a whole body MRI is carried out using an apparatus such as one of those described by Pykett (1982) Scientific American, 246:78-88 to locate and image cancerous tissues.

Also within the scope of the invention are kits comprising the protein ligand that binds to a particular target and to a serum albumin and instructions for diagnostic use, e.g., the use of the ligand to detect target-expressing cells, e.g., in vivo, e.g., by imaging a subject, e.g., a cancer patient. The kit can further contain a least one additional reagent, such as a label or additional diagnostic agent. For in vivo use the ligand can be formulated as a pharmaceutical composition.

The following non-limiting examples further illustrate aspects of the invention:

### EXAMPLE 1

### DX-954

DX-954 is a peptide that was isolated by phage display as a ligand that binds to VEGF-R2. DX-954 also binds to serum albumin since at high concentrations serum albumin prevents DX-954 from binding to VEGF-R2.

The amino acid sequence of DX-954 is: AGPTWCEDDWYYCWLFGTGGGK (SEQ ID NO: 1). The DX-954 peptide is acetylated at the amino terminus and amidated at the carboxy terminus.

### EXAMPLE 2

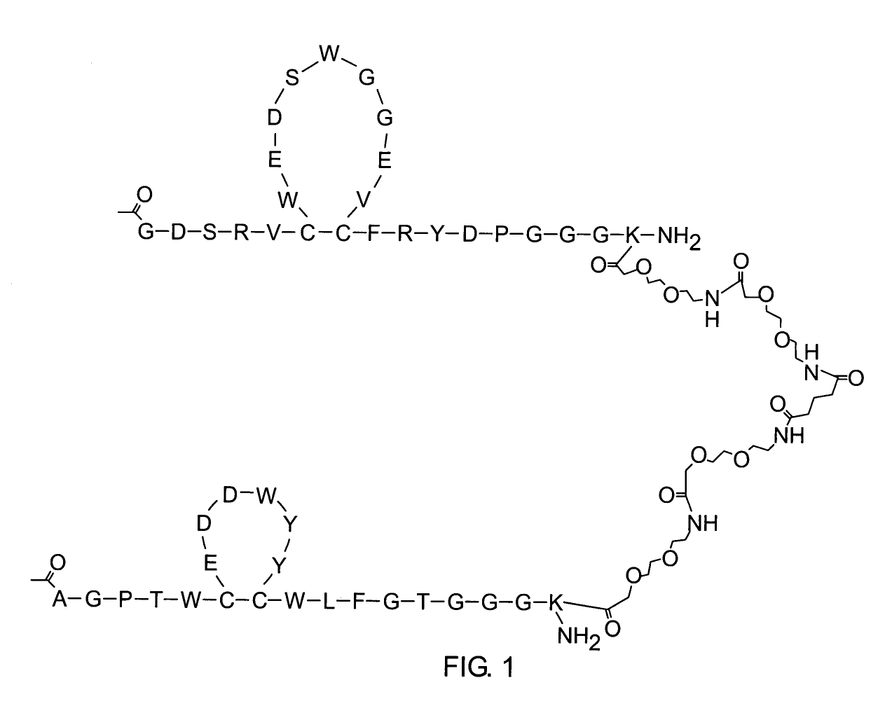

DX-1235, is a conjugate of DX-954 and another peptide DX-712, another VEG-FR2 binder. The amino acid sequence of DX-712 is: GDSRVCWEDSWGGEVCFRYDPGGGK (SEQ ID NO: 2). The structure of DX-1235 is shown in FIG. 1. The upper amino acid sequence in FIG. 1 corresponds to DX-712 (SEQ ID NO: 2; see also Example 2, below). The lower amino acid sequence in FIG. 1 corresponds to DX-954 (SEQ ID NO: 1, see also Example 1, below). The line connecting the two cysteines (“C”) in each amino acid sequence corresponds to a disulfide bond.

DX-1235 has a biphasic half-life for clearance from circulation. For the fast phase t_{half i}s about 2 minutes, and for the slow phase, thalf is about 30 minutes.

Serum samples from animals injected with DX-1235 were analyzed using size exclusion chromatography. DX-1235 was associated with fractions containing large molecular weight material. This finding is consistent with an interaction with HSA.

### EXAMPLE 3

U.S. Published application Ser. No. 2003/0,069,395 (U.S. Ser. No. 10/094,401) provides a number of peptides that bind to serum albumin. See, e.g., Table 8 of Ser. No. 2003/0,069,395. Motifs and amino acids that are over-represented in such peptides can be used to prepare a target-specific protein that also binds to a serum albumin. For example, such motifs and/or amino acids can be substituted into target-binding ligands at positions that are non-essential for binding.

The invention also provides other embodiments. For example, it may also be useful to develop peptides that bind to other serum components, e.g., components that may deliver a compound to a target region, e.g., fibrin, proteins on the surface of blood cells, immunoglobulins, and so forth. Other embodiments are provided in the summary and still others are within the scope of the following claims.

What is claimed:

1. A method of identifying a target-binding protein that binds to a predetermined target and to a serum albumin, the method comprising:
providing a plurality of diverse proteins; and
identifying one or a subset of members of the plurality which (1) interacts with a predetermined target, other than a serum albumin, and (2) binds to a serum albumin, thereby identifying a target-binding protein that binds to a predetermined target and to a serum albumin.

2. The method of claim 1 further comprising evaluating the in vivo half life of the identified member or at least some members of the subset.

3. The method of claim 1, wherein the plurality of diverse proteins comprise members of a display library.

4. The method of claim 3, wherein the identifying comprises screening a display library.

5. The method of claim 1, wherein the identifying comprises screening or selecting members of the plurality of diverse proteins that interact with the predetermined target, and then screening or selecting, from those members that interact with the predetermined target, for the one or the subset of members that also bind to serum albumin.

6. The method of claim 1, wherein the identifying comprises screening or selecting members of the plurality of diverse proteins that bind to the serum albumin, and then screening or selecting, from those members that bind to the serum albumin, for the one or the subset of members that also interact with the predetermined target.

7. The method of claim 1 wherein the serum albumin is human serum albumin.

8. The method of claim 1 wherein the predetermined target is an extracellular domain of a naturally occurring protein.

9. The method of claim 1 further comprising administering the identified member to a subject.

10. The method of claim 1 further comprising formulating the identified member or one or more members of the identified subset as a pharmaceutical composition.

11. The method of claim 1 wherein each diverse protein comprises a varied peptide of less than 30 amino acids in length.

12. The method of claim 11 wherein the varied peptide comprises less than 4 constant positions.

13. The method of claim 11 wherein the varied peptide comprises an intramolecular disulfide bond formed by two invariant cysteine residues.

14. A target-binding protein isolated by the method of claim 1 and that comprises a polypeptide that (1) interacts with a predetermined target, other than a serum albumin, and (2) binds to a serum albumin.

15. A method of identifying a target binding protein, the method comprising:
(a) providing a plurality of library members, each of which includes a diverse protein;
(b) identifying a subset of members of the plurality that binds to a predetermined target, other than serum albumin, or to a serum albumin;
(c) altering the sequence of at least one member of the subset to form an altered subset that includes a plurality of variants of the at least one member; and
(d) identifying one or a subset of members of the altered subset which binds to (1) the predetermined target if the identifying in (b) is to serum albumin or (2) the serum albumin, if the identifying in (b) is to the predetermined target, thereby identifying a target binding protein.

16. The method of claim 15 wherein the altering comprises comparing amino acid sequences of members of the subset, inferring at least one profile for at least some of the members, and preparing the altered library by varying positions not conserved in the at least one profile.

17. A method of identifying a target-binding protein that binds to a predetermined target and to a serum albumin, the method comprising:
providing an initial protein that specifically binds to a target compound;
preparing a plurality of variant proteins by altering one or more amino acid positions of the initial protein; and
selecting a target-binding protein that binds to a predetermined target and to a serum albumin from the plurality of variant proteins by evaluating one or more of the variant proteins for binding to the predetermined target and for binding to the serum albumin.

18. The method of claim 17 wherein the one or more variant proteins are evaluated by a method that comprises contacting the one or more variant proteins to immobilized serum albumin.

19. The method of claim 17 wherein preparing a plurality of variant proteins comprises altering a nucleic acid sequence that encodes the initial protein.

20. The method of claim 19 wherein preparing a plurality of variant proteins comprises constructing a display library.

21. The method of claim 17 wherein preparing a plurality of variant proteins comprises determining for the initial protein one or more amino acid positions that are non-essential for binding to the predetermined target and varying at least one of the non-essential positions.

22. The method of claim 17 wherein preparing a plurality of variant proteins comprises substituting at least one aromatic amino acid into an amino acid position of the initial protein.

23. The method of claim 17 wherein providing the initial protein comprises screening a display library.

24. An isolated peptide that specifically binds to a target molecule other than serum albumin with a K_{D} of less than 1 μM and that binds to a serum albumin.

25. The peptide of claim 24 wherein the peptide has a length of between 6 and 32 amino acids.

26. The peptide of claim 24 wherein the peptide binds to the serum albumin with a K_{D} that is greater than its K_{D} for the target molecule.

27. The peptide of claim 26 wherein t the peptide binds to the serum albumin with a K_{D} that is at least 5 fold greater than its K_{D} for the target molecule.

28. The peptide of claim 24 wherein the peptide has a half-life in vivo of at least 30 minutes in a mouse model system.

29. The peptide of claim 24 wherein the serum albumin is human serum albumin.

30. The peptide of claim 24 that comprises an intra-molecular disulfide bond.

31. The peptide of claim 24 that is attached to a cytotoxic moiety.

32. The peptide of claim 24 wherein the peptide comprises at least one aromatic an amino acid.

33. The peptide of claim 24 wherein the peptide comprises an aromatic di- or tri-peptide sequence.

34. The peptide of claim 24 wherein binding of the peptide to the target molecule and binding of the peptide to the serum albumin are mutually exclusive.

35. The peptide of claim 24 wherein residues of the peptide that mediate binding to the target molecule and residues that mediate binding to the serum albumin are co-extensive.

36. The peptide of claim 24 wherein the target molecule comprises an extracellular domain of a naturally occurring protein.

37. The peptide of claim 24 wherein the target molecule is selected from the group consisting of an integrin, CEA, VEGF-R2, and MUC1.

38. The peptide of claim 24 wherein the peptide and any conjugated moieties has a molecular weight of less than 4500 Daltons.

39. The peptide of claim 38 wherein the peptide and any conjugated moieties has a molecular weight of less than 3500 Daltons.

40. The peptide of claim 24 wherein the peptide binds to the target molecule with a K_{D} of less than 200 nM.

41. The peptide of claim 40 wherein the peptide binds to the target molecule with a K_{D} of less than 50 nM.

42. The peptide of claim 24 wherein the peptide binds to serum albumin with a K_{D} of between 50 nM and 50 μM.

43. The peptide of claim 38 wherein the peptide binds to serum albumin with a K_{D} of between 50 nM and 50 μM.

44. The peptide of claim 41 wherein the peptide binds to serum albumin with a K_{D} of between 50 nM and 50 μM.

45. An isolated peptide that specifically binds to a target molecule other than a serum albumin with a K_{D} of less than 1 μM and that binds to a human serum albumin with a K_{D} that is at least 5 fold greater than its K_{D} for the target molecule and that is between 50 nM and 50 μM, wherein the peptide has a length of between 6 and 32 amino acids, and wherein the peptide and any conjugated moieties has a molecular weight of less than 4500 Daltons, and wherein the peptide has a half-life in vivo of at least 30 minutes in a mouse model system.

46. A pharmaceutical composition comprising the peptide of claim 24 and a pharmaceutically acceptable carrier.

47. A pharmaceutical composition comprising the peptide of claim 45 and a pharmaceutically acceptable carrier.

48. An isolated nucleic acid comprising a sequence that encodes a polypeptide that comprises the peptide of claim 24.

49. An isolated nucleic acid comprising a sequence that encodes a polypeptide that comprises the peptide of claim 45.

50. A recombinant host cell that contains the nucleic acid of claim 48 and that can produce the polypeptide encoded by said nucleic acid.

51. A recombinant host cell that contains the nucleic acid of claim 49 and that can produce the polypeptide encoded by said nucleic acid.

52. A method of administering target-binding protein to a subject, the method comprising: administering the pharmaceutical composition of claim 46 to a subject.

53. The method of claim 52 further comprising evaluating a symptom of the subject.

54. The method of claim 52 further comprising imaging the subject.

55. The method of claim 52 further comprising evaluating clearance of the peptide from the subject

56. The method of claim 52 further comprising evaluating concentration of the peptide in the subject.

57. The method of claim 52 wherein the composition is administered as part of a regular dosage regimen, and the dosages are administered at least 24 hours apart.

58. A method of providing an agent, the method comprising: selecting a peptide agent which has been test for ability to bind to a target molecule other than a serum albumin and for ability to bind to serum albumin, thereby providing an agent.

59. The method of claim 58 further comprising administering the agent to a subject.


SEQUENCE LISTING (11 sequences)

SEQ ID NO: 1 - protein, 22 aa, Artificial Sequence
  Synthetically generated peptide
AGPTWCEDDWYYCWLFGTGGGK

SEQ ID NO: 2 - protein, 25 aa, Artificial Sequence
  Synthetically generated peptide
GDSRVCWEDSWGGEVCFRYDPGGGK

SEQ ID NO: 3 - protein, 585 aa, Homo sapiens [sequence omitted]

SEQ ID NO: 4 - protein, 608 aa, Mus musculus
MKWVTFLLLLFVSGSAFSRGVFRREAHKSEIAHRYNDLGEQHFKGLVLIAFSQYLQKCSYDEHAKLVQEVTDFAKTCVADESAANCDKSLHTLFGDKLCAIPNLRENYGELADCCTKQEPERNECFLQHKDDNPSLPPFERPEAEAMCTSFKENPTTFMGHYLHEVARRHPYFYAPELLYYAEQYNEILTQCCAEADKESCLTPKLDGVKEKALVSSVRQRMKCSSMQKFGERAFKAWAVARLSQTFPNADFAEITKLATDLTKVNKECCHGDLLECADDRAELAKYMCENQATISSKLQTCCDKPLLKKAHCLSEVEHDTMPADLPAIAADFVEDQEVCKNYAEAKDVFLGTFLYEYSRRHPDYSVSLLLRLAKKYEATLEKCCAEANPPACYGTVLAEFQPLVEEPKNLVKTNCDLYEKLGEYGFQNAILVRYTQKAPQVSTPTLVEAARNLGRVGTKCCTLPEDQRLPCVEDYLSAILNRVCLLHEKTPVSEHVTKCCSGSLVERRPCFSALTVDETYVPKEFKAETFTFHSDICTLPEKEKQIKKQTALAELVKHKPKATAEQLKTVMDDFAQFLDTCCKAADKDTCFSTEGPNLVTRCKDALA

SEQ ID NO: 5 - protein, 12 aa, Artificial Sequence
  template sequence
XXXCXXXXCXXX

SEQ ID NO: 6 - protein, 13 aa, Artificial Sequence
  template sequence
XXXCXXXXXCXXX

SEQ ID NO: 7 - protein, 14 aa, Artificial Sequence
  template sequence
XXXCXXXXXXCXXX

SEQ ID NO: 8 - protein, 15 aa, Artificial Sequence
  template sequence
XXXCXXXXXXXCXXX

SEQ ID NO: 9 - protein, 16 aa, Artificial Sequence
  template sequence
XXXCXXXXXXXXCXXX

SEQ ID NO: 10 - protein, 17 aa, Artificial Sequence
  template sequence
XXXCXXXXXXXXXCXXX

SEQ ID NO: 11 - protein, 18 aa, Artificial Sequence
  template sequence
XXXCXXXXXXXXXXCXXX